SEQUENCE LISTING (11 sequences)

SEQ ID NO: 1 - protein, 458 aa, Teladorsagia circumcincta
MPITKIHARQIYDSRGNPTVEVDLYTDKGVFRAAVPSGASTGVHEALELRDKDKKVHHGKGVLKAVANINEKIAPALIAKNFCVTQQRDIDQFMLALDGTENKSNLGANAILGVSLAVAKAGAVHKGMPLYKYLAELAGVSKVILPVPAFNVINGGSHAGNKLAMQEFMILPVGASSFHEAMRMGSEVYHHLKAEIKKRYGLDATAVGDEGGFAPNIQDNKEGLDLLKTAIDLAGYTGKISIGMDVAASEFYKEGKYDLDFKNPKSDPSKWLTGDQLAALYQTFIKEYPVVSIEDAFDQDDWDNWGKLKAATNIQLVGDDLTVTNPKRIRQAIDKKSCNCLLLKVNQIGSVTESIEAAKLSRSNGWGVMVSHRSGETEDTFIADLVVGLATGQIKTGAPCRSERLAKYNQLLRIEEELGKDAVYAGQNFRNPVAAAGAPVPYPDPLEPRAAAHHHHHH

SEQ ID NO: 2 - protein, 395 aa, Teladorsagia circumcincta
MRGSHHHHHHGMASMTGGQQMGRDLYDDDDKDHPFTMSVPPEIIKKIEDGYQTLQNAKDCHSLLKKYLTKEVVDQLKDKKTKLGATLWDVIQSGVANLDSGVGVYAPDAEAYTLFKPLFDPLIQDYHNGFSPSQKQPATDLGEGKTAQLVDLDPEGKYINSTRVRCGRSLQGYPFNPCLTEANYLEMEAKVKKIFENISDPELQGTYYPLDGMTKEVQNQLIKDHFLFKEGDRFLQAANACRYWPKGRGIFHNKNKTFLVWANEEDHLRIISMQNGGNVGQVLERLIKGVKIIQAQAPFSRDDRLGWLTFCPSNLGTTVRASVHIRLPKISAKPDFKKICDDLKLQIRGIHGEHSDSEGGVYDISNKARLGLTEFEAVKQMYDGVKHLIELEKKA

SEQ ID NO: 3 - protein, 424 aa, Teladorsagia circumcincta
MTMITQMELIGDSKVAIADGEVDAISMCQEIAHSYDQDNIDDAFMLVDLDVIFERFLLWKREMPMIEPFYAVKCNTDRVLVRTLAALGAGFDCASREEIDIVMDMGVRAEKIIYANPCKTRSFITHAKEKNVSMMTFDSVEELAKIAHLHPDAKMILRIAVSDPTARCPLNLKFGVDPVTKAPHLLVHAKELGVNVIGISFHVGSGCNDPTAFREALTHARHLTELGRGLGFDMNLVDLGGGYPGTLQQTSFEDIAAVIRSAVDEFLPPEFGVRLIAEPGRFFAAAPFTLVCNIIHATEVSAEKITKRPEDVDHRGFMYYVNDGVYGSFNCILFDHVDPVGAPLFDEIVEEYPSTIWGPTCDSLDKIEDQKMMRMMSVGEWIVYQNMGAYTCSASTTFNGFQRPNAVYVISRKNWARISTSPNV

SEQ ID NO: 4 - protein, 511 aa, Teladorsagia circumcincta
MVLDMDLFREEKGGNPELIRSSQRQRYSDPSIVDKVIELDQAWRKERFLLDVLNRQKNVLSKAIGEKMKKKEAQGTDENVDGSIISQLESLKMEDLSALTVTQIKKLRVLLDEKMNDTKVSMEQLEEDRHQSLIQIGNIVHHSVPVSDDEANNRVERTHGDITSRKKYSHVDLVVMIDGFDGERGTTVAGGRGYFLKGPLVFLEQAIIQLALQKLGEKGFTPLYTPFFMRKEVMQEVAQLNQFDEELYKVCGKGSEMLGDSSVDEKYLIATSEQPIAAFHRNEWIKESDLPIKYAGISTCFRQEVGSHGRDTRGIFRVHQFEKVEQFVICSPLNNESWKMFDEMIFNAEECCQLLGIPYQIMCIVSGELNNAASKKLDLEAWFPGSGAFRELVSCSNCTDYQARRLKVRYGMTKKMDGEVPFVHMLNATMCATTRVLCALLENYQTEDGITVPEVLHPFMPEKYRTFIPFVKPAPIDEEVKKKGGKAAAGAPVPYPDPLEPRAAAHHHHHH

SEQ ID NO: 5 - protein, 144 aa, Teladorsagia circumcincta
MPMVRVATNIPDKDVPPNFEERLTDILAESMNKPRTRIAVEVYAGQRIMHGGVRNPVVIIKIESIGALEPDKNIRHTERVTQLCQDVLHVPKDKVVISYFDLAPTNVGFGGTTVAAATVAAAGAPVPYPDPLEPRAAAHHHHHH

SEQ ID NO: 6 - protein, 401 aa, Teladorsagia circumcincta
MRGSHHHHHHGMASMTGGQQMGRDLYDDDDKDHPFTMASYSQPPPKEKEDELRGIANAIVAPGKGILAADESTGSMDKKMKGIGTENTEEQRRKYRQLLFTASPEMSKHISGVIMFHETFYQKCDDGTRFVDALKKQGIIPGIKVDKGVVPMAGTVGEGTTQGMDDLNARCAQYKKDGAQFAKWRCVHKISATTPSHMALVEIAEVLARYASICQQNGLVPIVEPEILPDGEHDIDRCRKITETVLSYCYRALNDHHVYLEGTLLKPNMVTAGQAFKGKKPSHDEIALATITALQRSVPAAVPGVVFLSGGQSEEDATLNLNAMNKLDTKKPWALTFSYGRALQASCMSKWGGKDENVKDAQAVFMQRAQANSLAALGKYSGDPNADKAASQSLFVANHAY

SEQ ID NO: 7 - protein, 341 aa, Teladorsagia circumcincta
MGKAKVGINGFGRIGRLVLRAAVEKDTVEVVSVNDPFINIDYMVYLFKYDSTHGRFKGSVSHEGDHLVVSKEGKSTHRIKVHNSKDPAEIPWGADGAEYIVESTGVFTTVDKASAHLKGGAKKVVISAPSADAPMFVMGVNNETYNASNNHIISNASCTTNCLAPLAKVIHDNFGIIEGLMTTVHATTATQKTVDGPSGKLWRDGRGAAQNIIPAATGAAKAVGKVIPALNGKLTGMAFRVPTPDVSVVDLTCRLEKPASMDDIKKVIKSASEGAMKGILGYTEDQVVSTDFLSDTHSSIFDAGACISLNPHFVKLISWYDNEYGYSHRVVDLLTYIASKA

SEQ ID NO: 8 - protein, 469 aa, Teladorsagia circumcincta
MAQAAKNWYQVVKAAPKGRFQGIKRDYQVEDVLKLRGSVEIEYTLATRGANKLWQLLHTEPFVPALGAQTGNQAVQMVRAGLKAIYLSGWQVAADANTAGDMYPDQSLYPANSGPELCRRINRSLRRADQIEAVEAEDYLAQRDWYAPIVADAEAGFGGALNCFELMKSYIEAGAAGVHFEDRLGSEKKCGHMGGKVLIPTAQHIRHLNAARLAADVCGAPTIVVARTDAESSRLLTSDVDERDHPYIDRQAGRTVEGFHRLKDSTALQYCIDRAINYAPYCDLIWMETSHPTIADAREFAEGVRKVYPDKMFAYNCSPSFNWKQHLSPTQLEKFQKELGALGFKYQFITLAGFHANSYSMFDLARNYKEKGMLAYSSLQQQEFAAEQHGYSAVKHQREVGTGYFDHISNAVTGGQSSTTALAGSTEEAQFHTATASSEDEEILAAAGAPVPYPDPLEPRAAAHHHHHH

SEQ ID NO: 9 - protein, 551 aa, Teladorsagia circumcincta
MTLTAPMAAGDEKILTPDALRFIKDLNKKFDGKRRELLKKRQQVQIEINDGVYFPDFSSETAHLREDMGWKGSEIPQDLQDRRVEITGPTDRKMVINALNSGANVFMADFEDSNTPSWRNQLEGQVNLYDAVRDNISYMHPTTKKEYTLNQKVAVLNVRPRGWHLPEKHVLIHNKPTSGSLFDFGLFVYHNAKALKDKGSGPYFYLPKLQNAEEAKLWAEVFAYSEDRLDLPRGTIKCTVLIEHLLATFQMNEIIYALKDHIVGLNCGRRDYIFSYIKTFQNHRKFLLPDRFQIGMTSPFMRAYSLLCIKTCHQRGIHAMGGMAAQIPIKNDDVANSKALALVHQDKEREATDGHDGTWVAHPGLVPIARKVFDDCMPSANQIEKQLQSFFVTNQELTAIPEGTRTDHGFRHNISVTLGYLDSWLRGVGCVPLYNLMEDAATAEISRSQLWQWLRHDARLEDGRTIDAQLVKQTIAAETERRLIRAGSVVSRLPEAAELLEKFALEEHMSDFLTLDAYDKLVSEGHAAAGAPVPYPDPLEPRAAAHHHHHH

SEQ ID NO: 10 - protein, 329 aa, Teladorsagia circumcincta
MVAETPVRRKVFINSAIAFVRQWDFDGIDIDWEYPSGPADVRNYASFISELRQACEAEATSSQKPRLLVTAAVSAGESTIDAGYDVPAIADHLDFILLMNYDFHGAWSTETGFNSPLYAREDMRESEKVWNIDWSANHWHQKGMAKEKIIIGIPTYGRGWTLKDKSNITVGAEGSPAKITPYTQEAGVASFYEFCEMLATGATRYWSSEQQVPYLVQGDQWWSYDDEESIANKMAWIKRNKYGGAFVWTLDFDDFNAKCSNSDGQLYPLISIIAKELGGVTIPKVSTCSTWGNMDDVVDKGLSIAAAGAPVPYPDPLEPRAAAHHHHHH

SEQ ID NO: 11 - protein, 230 aa, Teladorsagia circumcincta
MVHYRLLYFDGRGRAEVARQLFALANQEYVDVRITHEEWPKHKPEMPFGQLPVLDVDGKLLGQSHAINRYLARQFGFAGKSPFEEALVDAFADQYRDFYTEAQPYLYAVWGFVKGDVNALENEKFAPARDKFFNLMTKHLKASKSGFLVGDSVTWADLQLAELATFTEKYATLYVGFPEVKAHSEKVRSIPEIKKRIETRKNTPFAAAGAPVPYPDPLEPRAAAHHHHHH

(19) United States
(12) Patent Application Publication
(10) Pub. No.: US 2026/0263572 A1
(43) Pub. Date: Sep. 10, 2026

(54) NEMATODE VACCINE

(71) Applicant: AGRESEARCH LIMITED, Lincoin (NZ)

(72) Inventor: Saleh UMAIR, Palmerston North (NZ)

(21) Appl. No.: 19/167,281

(22) PCT Filed: Mar. 22, 2024
(86) PCT No.: PCT/NZ2024/050033

§ 371 Date: Sep. 19, 2025

(30) Foreign Application Priority Data
Mar. 27, 2023 (AU) 2023900839

(51) Int. Cl. A61K 39/00; A61K 39/39; A61P 37/04

(52) U.S. Cl. CPC A61K 39/0003; A61K 39/39; A61P 37/04; A61K 2039/552; A61K 2039/55566; A61K 2039/55577; A61K 2039/575

(57) ABSTRACT

The present invention is directed to a vaccine comprising recombinant antigens derived from the parasitic nematode Teladorsagia circumcincta, which will raise an immune response in farmed and wild ruminants that are susceptible or predisposed to infection by one or more nematode worm species.

### FIELD OF THE INVENTION

The present invention relates to a vaccine comprising antigens which stimulate or boost acquired immunity against infection by parasitic nematodes, particularly in farmed or wild ruminants such as sheep, cattle, goats, deer, buffalo, bison, camelids, llamas, etc.

### BACKGROUND OF THE INVENTION

Parasitic nematode worm infection is one of the biggest health problems for farmed ruminants worldwide. Parasitic worm infections are harmful to a host animal for many reasons. For example, they deprive the host of food, damage internal tissues and organs, cause anaemia, weight loss, diarrhoea, dehydration and loss of appetite. Such parasitic infections cause costly production losses and if left untreated, animals can die causing further economic loss to farmers.

Currently, farmers rely on the use of anthelmintic agents (such as benzimidazoles, levamisole, morantel, monepantel, oxfendazole or ivermectin) to control parasitic nematodes, however resistance of parasites to one or more of these agents is now widespread. Indeed, recent industry-funded surveys in New Zealand found that 64% of sheep farms and 94% of beef farms now have parasites that are resistant to at least one of these anthelmintics.

Such resistance in terms of control and productivity losses is estimated to cost the New Zealand livestock industry around $700 million annually.

Alternative methods of controlling the effect of on-farm parasite infections have been proposed and includes altered grazing management, use of nematode trapping fungi, dietary supplements, selective breeding of animals for host resistance and vaccines.

Attempts to develop recombinant vaccines against parasitic nematodes have met with limited success and to date there are no commercial recombinant vaccines available for any nematode parasites. However, the development of such a vaccine is viewed by the industry as a solution to the resistance problem.

One target for a protective vaccine is against essential worm metabolic enzymes. Parasitic nematode larvae grow rapidly and adult worms lay large numbers of eggs, both requiring highly active nitrogen and energy metabolism. Essential worm enzymes involved in these pathways, and which are not present in the host, are therefore potential targets for controlling parasites. Essential enzymes involved in blood digestion and other pathways critical to the life cycle of the worm could also be targeted either alone or as multiple targets.

Vaccination with antigens comprising such metabolic enzymes would in theory generate circulating host antibodies which would bind to and disrupt the function of the essential parasitic metabolic enzymes, hopefully leading to a substantially reduced worm burden and faecal egg count (FEC).

A vaccine (Barbervax) based on an extract of adult H. contortus has recently been released commercially in Australia. While the vaccine is effective in protecting sheep against infection, there are a number of real or potential issues with its use. First, the vaccine does not provide any long-term protection against infection, and as a result needs to be applied on several occasions over the period of risk. Second, there is significant risk of degradation of the native antigen should it be subjected to high temperatures in the field. Third, as the antigen is extracted from worms derived from donor sheep, there may be a risk of cross-contamination with infectious agents such as viruses.

Recombinant antigens would overcome these issues. One group have successfully trialed a T. circumcincta vaccine comprising eight recombinant proteins (APY-1; MEP-1; ES20; CF-1; TGH-2; ASP-1; MIF-1; and SAA-1). These recombinant proteins were carefully chosen using a tripartate approach. First antigens were chosen that were excreted/secreted during the critical phase of worm growth; then they chose SAA-1, an immunogenic homologue of a protective antigen from canine hookworm; and finally, they selected antigens that were potentially immunosuppressive molecules (WO2013/117912). While an antigen with all eight recombinant proteins showed reduced worm burdens and FEC similar to those following vaccination with detergent extracts of T. circumcincta, a later publication suggests that these recombinant antigens induce a suboptimal antibody response due to differences between native antigens and their recombinant versions because of variations in structure and/or post-translational modifications (Longhi-Browne, 2014).

Because of such problems in recombinant proteins technology, attempts to make commercial vaccines from recombinant antigens have so far failed. There is therefore a need in the art to provide such recombinant vaccines.

It is an object of the present invention to go some way towards overcoming this need and/or to provide the public with a useful choice.

### SUMMARY OF THE INVENTION

The present invention is directed to a vaccine comprising recombinant antigens derived from the parasitic nematode Teladorsagia circumcincta, which will raise an immune response in farmed and wild ruminants that are susceptible or predisposed to infection by one or more nematode worm species. The recombinant antigens used in the invention are conserved among species of nematode worms so that the vaccine will provide protection against multiple types of nematode worms.

In a first embodiment, the invention provides a composition or vaccine composition comprising the recombinant T. circumcincta antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) aldolase; and
(vii) glyceraldehyde-3-phosphate dehydrogenase (GAPDH), or antigenic fragments thereof together with a veterinary acceptance carrier or diluent.

The composition or vaccine composition may further comprise one or more recombinant T. circumcincta antigens selected from the group consisting of:
(viii) isocitrate lyase (ICL);
(ix) malate synthase;
(x) chitinase (Cht); and
(xi) glutathione S-transferase (GST)

Preferably, the composition or vaccine composition comprises at least one, at least two, at least three, or at least four, of the antigens (iv) to (xi), above.

In a second embodiment, the invention provides a composition or vaccine composition comprising the T. circumcincta recombinant antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) aldolase;
(vii) glyceraldehyde-3-phosphate dehydrogenase (GAPDH);
(viii) isocitrate lyase (ICL);
(ix) malate synthase;
(x) chitinase (Cht); and
(xi) glutathione S-transferase (GST)
or antigenic fragments thereof, together with a veterinary acceptable carrier or diluent.

The composition or vaccine composition may further comprise an adjuvant, such as: alum, Quil A, Freund's complete adjuvant, Freund's incomplete adjuvant, lipopolysacharride, monophosphoryl lipid A, montanide, lipovant, bacterial flagellin, adjuvant 65, gamma inulin, algammulin, imiquimod, guardiquimod, murimyl dipeptide, etc.

The composition or vaccine composition may further comprise a carrier such as: a chitin-based slow release compound (sol-gel), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes, cochleate delivery vehicles, etc.

In a third embodiment, the invention provides a method of reducing parasitic nematode worm burden in a farmed or wild ruminant animal, said method comprising administering an effective amount of the composition or vaccine composition of the invention to said ruminant animal on one or more occasions, whereby parasitic worm burden reduction is measured by a reduced faecal egg count (FEC), and/or an increase in expulsion of larvae and/or adult nematode worms.

In a fourth embodiment, the invention provides a method of inducing an immune response in a farmed or wild ruminant animal to treat or protect said animal against infection by parasitic nematodes, said method comprising administering an effective amount of the composition or vaccine composition of the invention to said animal on one or more occasions, wherein induction of an immune response is measured by the presence of protective antibodies against one or more specific antigens present in said composition or vaccine composition.

In a fifth embodiment, the invention provides a method of stimulating or boosting acquired immunity in a farmed or wild ruminant animal to treat or protect said animal against infection by parasitic nematodes, said method comprising administering an effective amount of the composition or vaccine composition of the invention to said animal on one or more occasions, wherein stimulation or a boost of said acquired immunity is measured by one or more of: the presence of protective antibodies against one or more specific antigens present in said composition or vaccine composition; an increased level of cytokines; a reduced FEC; and/or expulsion of larvae and/or adult nematodes.

In a sixth embodiment, the invention provides a method of treating or preventing a nematode infection in a farmed or wild ruminant animal comprising administering an effective amount of said composition or vaccine composition to said animal.

In a seventh embodiment, the invention provides a use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase, and (vii) glyceraldehyde-3-phosphate dehydrogenase (GAPDH), or antigenic fragments thereof, in the manufacture of a composition or vaccine composition for reducing nematode parasitic worm burden in a farmed or wild ruminant animal.

In an eighth embodiment, the invention provides a use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase, and (vii) glyceraldehyde-3-phosphate dehydrogenase (GAPDH), or antigenic fragments thereof in the manufacture of a composition or vaccine composition for stimulating or boosting acquired immunity in a farmed or wild ruminant animal to treat or protect said animal against infection by parasitic nematodes.

In a ninth embodiment, the invention provides a use of the recombinant H. contortus antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase, and (vii) glyceraldehyde-3-phosphate dehydrogenase (GAPDH), or antigenic fragments thereof, in the manufacture of a composition or vaccine composition for treating or preventing a nematode infection in a farmed or wild ruminant animal.

In a tenth embodiment, the invention provides a use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase, and (vii) glyceraldehyde-3-phosphate dehydrogenase (GAPDH), or antigenic fragments thereof, in the manufacture of a composition or vaccine composition for inducing an immune response in a farmed or wild ruminant animal to treat or protect said animal against infection by parasitic nematodes.

The composition or vaccine composition used in these embodiments of the invention may further comprise one or more of antigens (viii)-(xi), above.

The farmed or wild ruminant animal is selected from the group consisting of sheep, cattle, goat, deer, buffalo, bison, camelids, llamas etc. The farmed or wild ruminant animals are preferably young animals, less than one year old, i.e. lambs, calves, kid goats etc. In one aspect, the farmed or wild ruminant animal is less than 6 months old. In a further aspect, the farmed or wild ruminant animal is at least 3 months old.

The parasitic nematodes treatable by the methods of this invention include Trichostrongylus colubriformis, Haemonchus contortus, Haemonchus placei, Ostertagia (Teladorsagia) circumcincta, Cooperia curticei, Nematodirus spathiger, Trichostrongylus axi, Trichostrongylus vitrinus, Ostertagia ostertagia, Cooperia oncophera, Nematodirus brasiliensis, Dictyocaulus eckerti, Strongylus vulgaris, Toxascaris vitolorum, Nematodirus filicollis, Ashworthius sidemi, Mecistocirrus digitatus, Bunostomum trigonocephalum, Trichuris discolor, Toxacara vitulorum, etc.

### BRIEF DESCRIPTION OF THE DRAWINGS

The invention will now be described in more detail with reference to the accompanying drawings in which:

FIGS. 1a-c: shows the degree of homology of the metabolic enzymes AK (FIG. 1a), EN (FIG. 1b) and ODC (FIG. 1c) across nematode species as follows:

FIG. 1a shows comparison of predicted arginine kinase amino acid sequences from the members of the Strongylida: Haemonchus contortus (Genebank Accession No. AFT82971), Teladorsagia circumcincta (AFT82970), Necator americanius (ETN81593) and Ancylostoma ceylanicum (EYC23758; EYC23757; EYB91576), and from members of the Rhabditidae: Caenorhabditis elegans (CAB00062; NP509217; NP507054; CCD73398; CCD79398), Caenorhabditis briggsae (CAP24981; CAP24932), Caenorhabditis brenneri (EGT52941; EGT41918) and Caenorhabditis remanei (EFP12066; EFO86450; EFO82749);

FIG. 1b shows comparison of predicted enolase amino acid sequences from members of the Strongylida: Haemonchus contortus (Genebank Accession No. AGC24386; ADK47524; CDJ96217), Teladorsagia circumcincta (deduced from T. circumcincta genome sequence), Mecistocirrus digitatus (BAN67669), Ancylostoma ceylanicum (EYB81234), Angiostrongylus cantonensis (AGO81688), Necator americanus (ETN80540), and from members of the Rhabditidae: Caenorhabditis elegans (NP495900; NP871916; NP001022349), Caenorhabditis brenneri (EGT35078) Caenorhabditis briggsae (CAP23453), and Caenorhabditis remanei (EFO85696);

FIG. 1c shows comparison of predicted ornithine decarboxylase amino acid sequences from members of the Strongylida: Haemonchus contortus (Genebank Accession No. AAC27893), Teladorsagia circumcincta (AGH70348) and Ancylostoma ceylanicum (EYC11973; EYC11971; EYC11970), and from members of the Rhabditidae: Caenorhabditis elegans (P41931), Caenorhabditis briggsae (CAP36352), Caenorhabditis brenneri (EGT47038) and Caenorhabditis remanei (EFP05480).

The alignments in FIGS. 1a-c were performed using the Muscle alignment option in Geneious 5.6.5 (Biomatters Ltd) with the Blosum 62 similarity matrix used to determine 100% similar residues (shaded). The consensus sequence shown is for the most common residue with the fewest ambiguities;

FIGS. 2a-g: show serum antibody response against 7 recombinant antigens-AK, EN, MIF-2, SRS-2, GAPDH, ODC, and Aldolase (7AgV), in sheep when Quil A was used as adjuvant (G1/G2) or when Montanide was used as adjuvant (G3/G4), G2 and G4 groups being adjuvant only, i.e. control groups;

FIG. 3: show saliva IgA response in sheep treated with 7AgV compared to controls in two adjuvants (Quil A vs Montanide);

FIG. 4: shows total (both male and female) worm burden of sheep treated with the 7AgV antigen compared to controls in two adjuvants (Quil A vs Montanide);

FIG. 5: shows the predicted means of faecal egg count (FEC) in sheep treated with 7AgV compared to control groups in two adjuvants (Quil A vs Montanide);

FIG. 6: shows the worm burden of sheep treated with 7AgV, 8aAgV, 8bAgV or 11AgV in Montanide adjuvant compared to control groups, in a second sheep trial;

FIG. 7a: shows the predicted means of FEC in sheep treated with 7AgV, 8aAgV, 8bAgV and 11AgV in Montanide adjuvant compared to control groups;

FIG. 7b: shows the serum IgG levels in the pooled serum samples of the 11AgV group and control group throughout the course of the trial;

FIG. 8: shows IgG levels in serum samples of sheep treated with 11AgV after with challenge with susceptible and resistant worms and with lower antigen dose compared to control group, in a third sheep trial;

FIG. 9: shows predicted means of faecal egg count in vaccine and control groups;

FIG. 10: shows total worm counts in control and treatment groups;

FIG. 11: shows IgG levels in serum samples of calves treated with 11AgV compared to control group, in a first calf trial;

FIG. 12: shows mean faecal egg count in vaccine and control groups;

FIG. 13: shows male, female and total worm count in vaccine and control groups;

FIG. 14: shows IgG levels in serum samples of deer treated with 11AgV compared to control group, in a first deer trial;

FIG. 15: shows IgA levels in saliva samples of deer treated with 11AgV compared to control group;

FIG. 16: shows means of faecal egg count in vaccine and control groups;

FIG. 17: shows total worm counts in vaccine and control groups;

FIG. 18: shows IgG levels in serum samples against recombinant AK of deer treated with 3AgV, 7 AgV and 11AgV in two adjuvants (Montadide vs QuilA/Sol gel) compared to control groups, in a second calf trial;

FIG. 19: shows IgG levels in serum samples against recombinant GAPDH of deer treated with 3AgV, 7AgV and 11AgV in two adjuvants compared to control groups;

FIG. 20: shows means of faecal egg count in vaccine and control groups;

FIG. 21: shows means of calf body weights in vaccine and control groups;

FIG. 22: shows total worm counts in control and vaccine groups;

FIG. 23: shows total arrested L4 counts in control and vaccine groups;

FIG. 24: shows the predicted means of faecal egg count (FEC) in calves treated with 7AgV and 11AgV compared to control groups in a third calf trial;

FIG. 25: shows total worm counts in vaccine and control and treatment groups;

FIG. 26: shows total arrested L4 counts in control and vaccine groups; and

FIG. 27: shows IgG levels in serum samples tested against all 11 recombinant antigens compared to control groups.

### DETAILED DESCRIPTION

Nematode worm infestation of farmed and wild ruminants around the world and Teladorsagia circumcincta is a major cause of parasitic gastroenteritis in small ruminants in cool temperate regions.

There is currently no protein-based vaccine against T. circumcincta available commercially.

The present invention provides for the first time an effective vaccine against nematode worm infestation in farmed and wild ruminants comprising a mixture of recombinant antigens.

The recombinant antigens correspond to T. circumcincta metabolic enzymes that are obligatory for worm survival.

The term “antigen” used herein means a molecule that provokes an immune response involving antibody production.

Without wishing to be bound by theory it is thought that antibodies produced as a result of immunisation with the vaccine composition of the invention act in two main ways. Firstly, with nematodes such as T. circumcincta that cause damage to the gut lining, the worms will be continually bathed in inflammatory exudate, some of which they ingest. It is therefore hypothesised that the worms will ingest the antibodies. Ingested antibodies will bind to target antigens, in this case, essential metabolic enzymes present in the intestinal wall of the nematode or secreted into the intestine cavity, thereby inhibiting their activity resulting in weakness of the worms which are then removed from the gut of the host animal by peristalsis. Other blood sucking nematodes, such as Haemonchus, will also ingest the antibodies in the host blood during feeding. In addition, the antibodies generated by the vaccine composition of the invention, include antibodies directed against antigens found in worm somatic tissue and/or secretory/excretory products affecting the worms ability to survive in the host intestine. The worms become weak and are expelled.

Efficacy of the vaccine composition of the invention can be measured by an increase in expulsion of larvae and/or adult nematodes, and/or by a reduced faecal egg count (FEC), as well as by the presence of one or more protective antibodies targeted by the antigens present in the vaccine composition.

The antigens present in the composition or vaccine composition of the present invention comprise (i) recombinant T. circumcincta enolase (EN), (ii) recombinant T. circumcincta arginine kinase (AK), (iii) recombinant T. circumcincta ornithine decarboxylase (ODC), (iv) recombinant T. circumcincta aldolase; (v) recombinant T. circumcincta seryl tRNA synthetase (SRS-2), (vi) recombinant T. circumcincta macrophage migration inhibitory factor 2 (MIF-2), and (vii) recombinant T. circumcincta glyceraldehyde-3-phosphate dehydrogenase (GAPDH), or antigenic fragments thereof.

Enolase is an enzyme involved in the glycolytic pathway and is a secreted enzyme forming part of the excretory/secretory (ES) complex. Enolase plays a vital role in the metabolism of nematode worms (Han et al, 2012). Arginine kinase is thought to be present in the cells lining the parasite gut and plays a vital role in the maintenance of ATP levels. Ornithine decarboxylase catalyses the conversion of ornithine into putrecine and is a rate-limiting enzyme in polyamine biosynthesis. Inhibition of ODC results in a loss of cell proliferation. Aldolase and GAPDH are essential enzymes involved in energy metabolism. Seryl tRNA synthetase is involved in translation and macrophage migration inhibitory factor 2 is involved in innate and acquired worm immunity. Helminth chitinases are induced during helper type responses and contribute to helminth immunity. Isocitrate lyase ansd malate synthase are key glycolytic enzymes. Glutathione transferase protects parasites against oxidative stress, and toxic and carcinogenic effects of endogenous substances. Inhibition of these enzymes by antibodies raised in response to inoculation of the vaccine of the present invention is shown for the first time to result in a significant reduction in fecal egg count (FEC), worm burden and other symptoms of T. circumcincta infestation in sheep.

Enolase and arginine kinase are highly conserved enzymes across nematode worm species so that the vaccine of the present invention is anticipated to be effective against a host of nematodes that infect farmed and wild ruminants including Bunostomum, Strongylus, Trichostrongylus, Haemonchus, Teladorsagia (Ostertagia), Toxascaris, Nematodirus, Trichuris, Dictyocaulus, Toxocara, Strongyloides, Cooperia, Ashworthius and Mecistrocirrus.

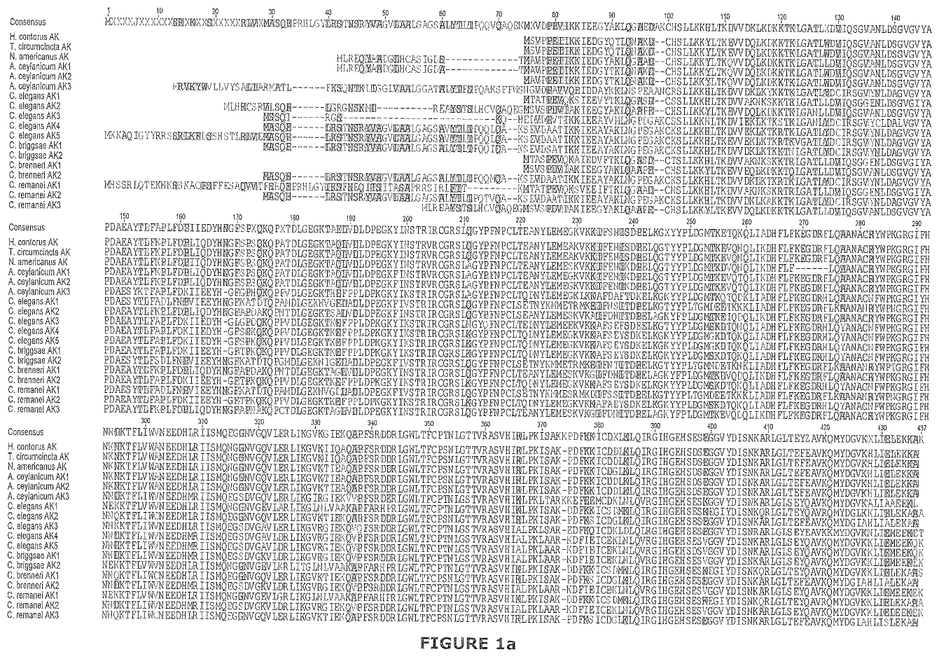

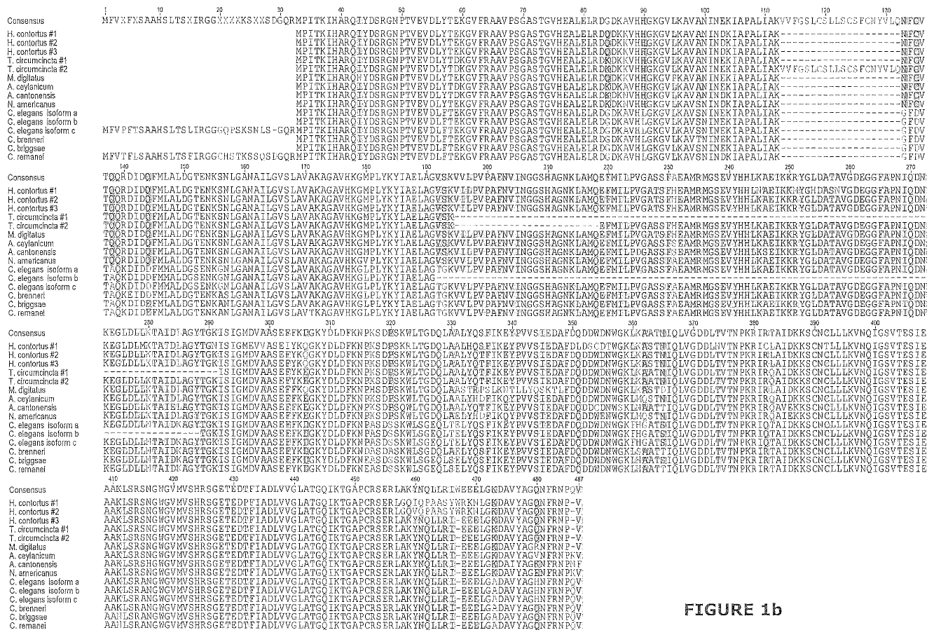

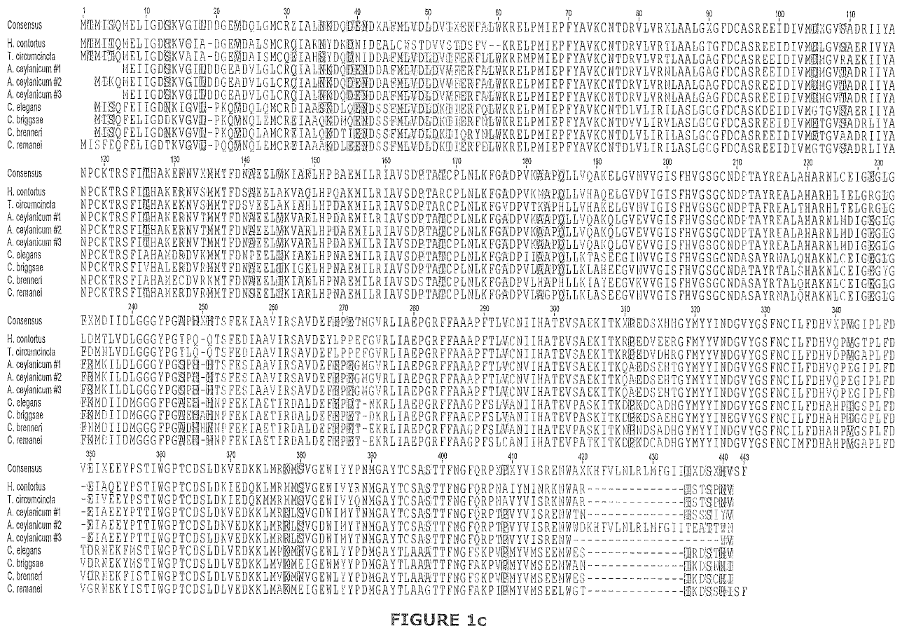

The degree of homology of EN, AK, ODC, SRS-2, MIF-2, aldolase, GAPDH, ICL, malate synthase, Cht and GST across nematode worm species is shown in FIGS. 1a-1c, and in Table 1, below.

As will be understood by a skilled worker, it is expected that, as there is such high conservation of EN and AK across species, the vaccine containing these T. circumcincta antigens will raise antibodies that will recognise EN and AK of other nematode species and work in the same way to block enzyme activity and so impact detrimentally on worm survival.

TABLE 1
The % of identical amino acid residues shared with T. circumcincta
arginine kinase (T. circumcincta AK; GenBank Accession No.
JX422017), T. circumcincta enolase (T. circumcincta GenBank Accession
No. KX452941) and T. circumcincta ornithine decarboxylase
(T. circumcincta; GenBank Accession No KC484698).
% homologies for T. circumcincta

| | |
|---|---|
| Species | % Identity |

arginine kinase

| | |
|---|---|
| T. circumcincta AK | |
| H. contorus AK | 96 |
| N. americanus AK | 94 |
| A. ceylanicum AK1 | 95 |
| A. ceylanicum AK2 | 95 |
| A. ceylanicum AK3 | 70 |
| C. elegans AK1 | 77 |
| C. elegans AK2 | 87 |
| C. elegans AK3 | 73 |
| C. elegans AK4 | 71 |
| C. elegans AK5 | 72 |
| C. briggsae AK1 | 71 |
| C. briggsae AK2 | 72 |
| C. brenneri AK1 | 88 |
| C. brenneri AK2 | 71 |
| C. remanei AK1 | 71 |
| C. remanei AK2 | 72 |
| C. remanei AK3 | 88 |

enolase

| | |
|---|---|
| T. circumcincta | |
| H. contortus #1 | 96 |
| H. contortus #2 | 94 |
| H. contortus #3 | 91 |
| M. digitatus | 84 |
| A. ceylanicum | 86 |
| A. cantonensis | 86 |
| N. americanus | 86 |
| C. elegans isoform a | 83 |
| C. elegans isoform b | 63 |
| C. elegans isoform c | 83 |
| C. brenneri | 84 |
| C. briggsae | 83 |
| C. remanei | 83 |

ornithine decarboxylase

| | |
|---|---|
| T. circumcincta | |
| H. contortus | 86 |
| A. ceylanicum #1 | 79 |
| A. ceylanicum #2 | 73 |
| A. ceylanicum #3 | 77 |
| C. elegans | 64 |
| C. briggsae | 64 |
| C. brenneri | 62 |
| C. remanei | 62 |

| | % Identity |
|---|---|
| Macrophage inhibition Factor-2 | |
| TcMIF-2 | |
| HcMIF-2 | 84 |
| Heligmosomoides polygyrus MIF-2 | 80 |
| Ancylostoma caninum MIF-2 | 75 |
| Parastrongyloides trichosuri MIF-2 | 55 |
| Caenorhabditis elegans MIF-2 | 54 |
| Caenorhabditis briggsae MIF-2 | 59 |
| Brugia malayi MIF-2 | 29 |
| Onchocerca volvulus MIF-2 | 29 |
| Oesophagostomum dentatum MIF-2 | 73 |
| Ancylostoma ceylanicum MIF-2 | 76 |
| Ascaris suum MIF-2 | 30 |
| Caenorhabditis elegans MIF-1 | 21 |
| Caenorhabditis elegans MIF-3 | 28 |
| Caenorhabditis elegans MIF-4 | 22 |
| Mus musculus MIF | 37 |
| Gallus gallus MIF | 35 |
| Ovis aries MIF | 33 |
| Bos taurus MIF | 44 |
| Homo sapiens MIF | 35 |
| Chitinase | |
| T. circumcincta CHT | |
| H. contortus CHT | 87 |
| H. placei CHT | 88 |
| A. ceylanicum CHT | 80 |
| N. americanus CHT | 81 |
| N. brasiliensis CHT | 81 |
| O. dentatum CHT | 80 |
| C. elegans CHT | 65 |
| Aldolase | |
| T. circumcincta | |
| Haemonchus contortus | 94 |
| Caenorhabditis elegans | 78 |
| Caenorhabditis elegans | 61 |
| Caenorhabditis briggsae | 77 |
| Drosophila melanogaster | 79 |
| Ancylostoma ceylanicum | 65 |
| Clonorchis sinensis | 62 |
| Echinococcus granulosus | 64 |
| Necator americanus | 86 |
| Schistosoma japonicum | 62 |
| Opisthorchis viverrini | 63 |
| Heterodera glycines | 63 |
| Iso-citrate lyase and malate synthase | |
| T. circumcincta ICL-MS | |
| A. ceylanicum ICL-MS | 92 |
| A. caninum ICL-MS | 88 |
| H. placei ICL-MS | 90 |
| H. contortus ICL-MS | 89 |
| N. americanus ICL-MS | 87 |
| C. elegans ICL-MS | 83 |
| D. viviparus ICL-MS | 83 |
| C. briggsae ICL-MS | 83 |
| S. stercoralis ICL-MS | 72 |
| T. canis ICL-MS | 71 |
| GST | |
| Teladorsagia circumcincta GST | |
| Haemonchus contortus GST | 64 |
| Ancylostoma caninum GST | 57 |
| Ascaris lumbricoides GST | 24 |
| Fasciola gigantica GST | 24 |
| Fasciola hepatica GST | 22 |
| Onchocerca volvulus GST | 21 |
| Caenorhabditis elegans GST | 23 |
| Caenorhabditis elegans GST | 22 |
| Ancylostoma duodenale GST | 32 |
| Dirofilaria immitis GST | 58 |
| Schistosoma japonicum GST | 20 |
| Trichinella spiralis GST | 26 |
| Schistosoma bovis GST | 18 |
| Schistosoma mansoni GST | 18 |
| GAPDH | |
| T. circumcincta GAPDH | |
| H. contortus GAPDH | 93 |
| B. malayi GAPDH | 86 |
| S. mansoni GAPDH | 71 |
| S. bovis GAPDH | 71 |
| S. japonicum GAPDH | 71 |
| F. hepatica GAPDH | 69 |
| D. viviparus GAPDH | 93 |
| C. elegans GAPDH | 84 |
| C. briggsae GAPDH | 84 |
| A. suum GAPDH | 82 |
| O. volvulus GAPDH | 84 |
| seryl tRNA synthetase | |
| T. circumcincta SRS-2 | |
| H. contortus SRS-2 | 94 |
| H. placei SRS-2 | 94 |
| H. polygyrus SRS-2 | 91 |
| A. ceylanicum SRS-2 | 90 |
| N. brasiliensis SRS-2 | 89 |
| N. americanus SRS-2 | 88 |
| C. remanei SRS-2 | 77 |
| L. loa SRS-2 | 73 |
| M. musculus SRS-2 | 68 |

Examples of specific nematode worm species that the vaccine of the present invention can be used to target include Trichostrongylus colubriformis, Haemonchus contortus, Haemonchus placei, Ostertagia (Teladorsagia) circumcincta, Cooperia curticei, Nematodirus spathiger, Trichostrongylus axei, Trichostrongylus vitrinus, Ostertagia ostertagia, Cooperia oncophera, Nematodirus brasiliensis, Dictyocaulus viviparus, Dictyocaulus eckerti, Strongylus vulgaris, Taxascaris vitulorum, Nematodirus filicollis, Ashworthius sidemi, Mecistocirrus digitatus, Bunostomum trigonocephalum, Trichuris discolor, Toxacara vitulorum, etc.

Preferably the parasitic nematode is Teladorsagia circumcincta.

In addition to (i) EN, (ii) AK, (iii) ODC, (iv) SRS-2, (v) MIF-2, (vi) aldolase; and (vii) GAPDH, the composition or vaccine composition of the present invention can further comprise one or more recombinant T. circumcincta antigens selected from the group consisting of:
(viii) isocitrate lyase (ICL);
(ix) malate synthase;
(x) chitinase (CHT); and
(xi) glutathione S-transferase (GST)
or antigenic fragments thereof.

Preferably, the composition or vaccine composition of the invention further comprises at least one, at least two, at least three, or all four of the antigens (viii)-(xi), above.

The % sequence identity for EN, EK and ODC across species of nematode worms is shown in FIGS. 1a-1c and in Table 1, above. Homology for the remaining antigens (iv)-(xi) is not shown.

It was expected that a composition or vaccine composition of the invention having additional antigens to (i) EN (ii) AK, (iii) ODC, (iv) SRS-2, (v) MIF-2, (vi) aldolase, and (vii) GAPDH above, will result in a stronger immunogenic response and improved reduction in FEC and worm burden due to at least an additive effect of each individual antigen.

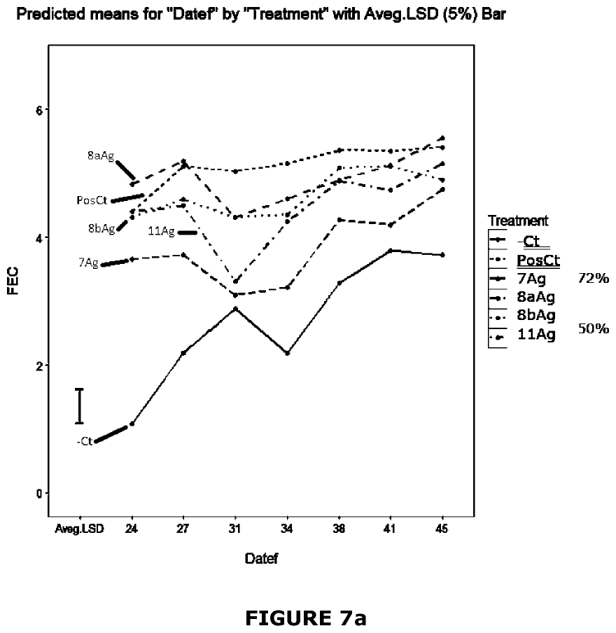

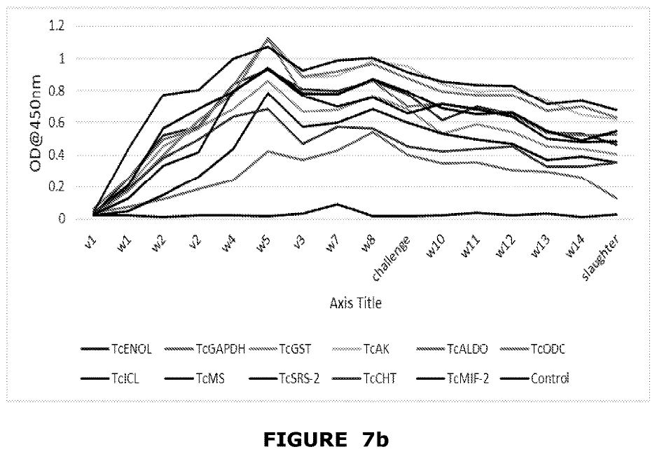

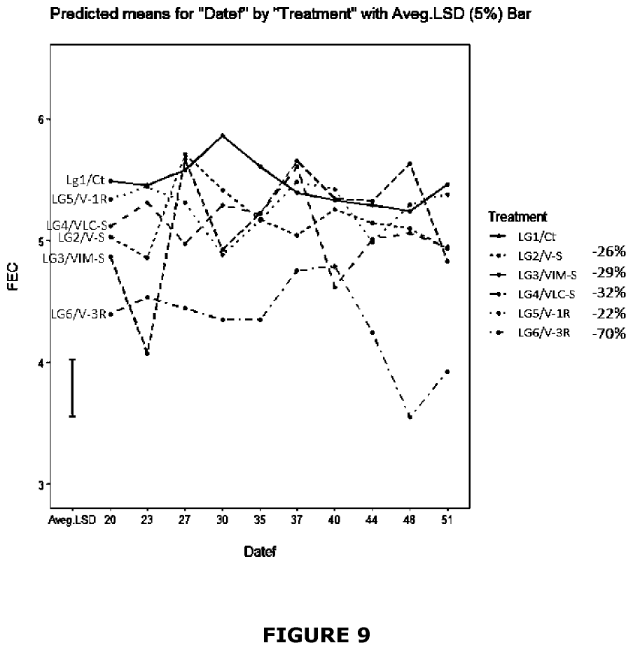

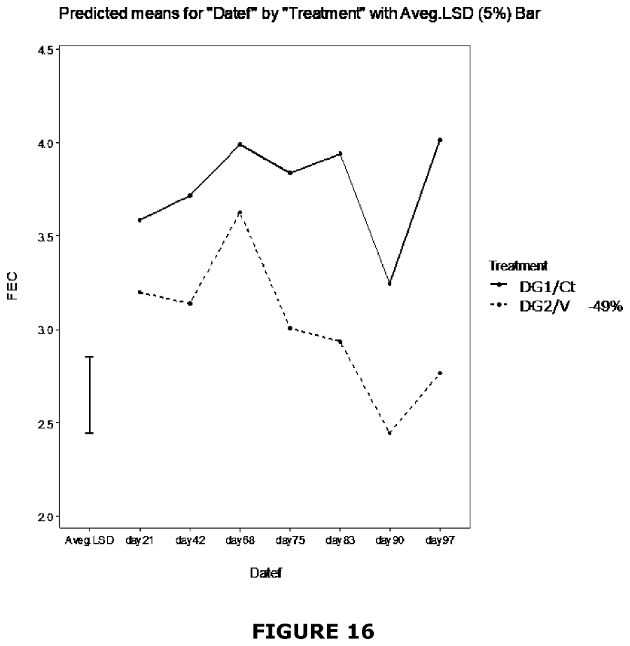

However, both the 7 and 11 antigen vaccines generally resulted in similar reductions in FEC in both sheep and deer (72% reduction in sheep using 7AgV and 50% reduction using 11AgV, see FIG. 7); 70% reduction is sheep using the 11AgV (see FIG. 9); and 49% reduction in FEC in deer (see FIG. 16). However, a 50-70% reduction of FEC is still considered to be a significant reduction and proves efficacy of the 7 and 11AgV vaccine compositions of the invention.

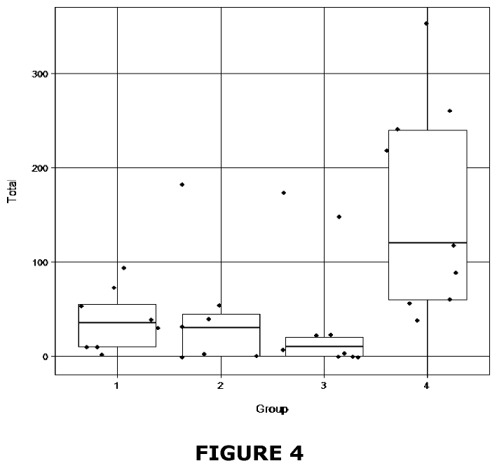

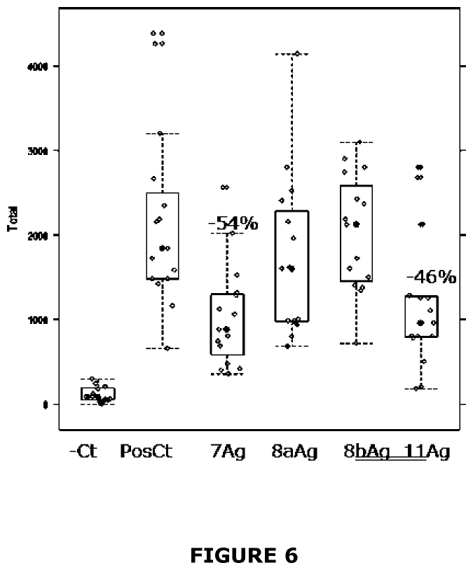

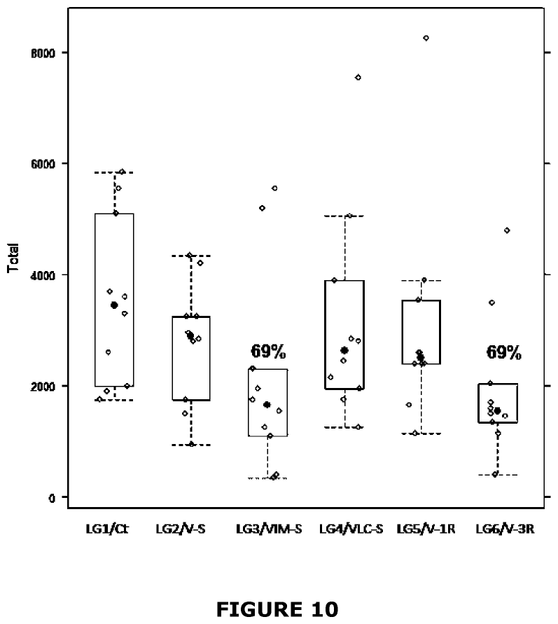

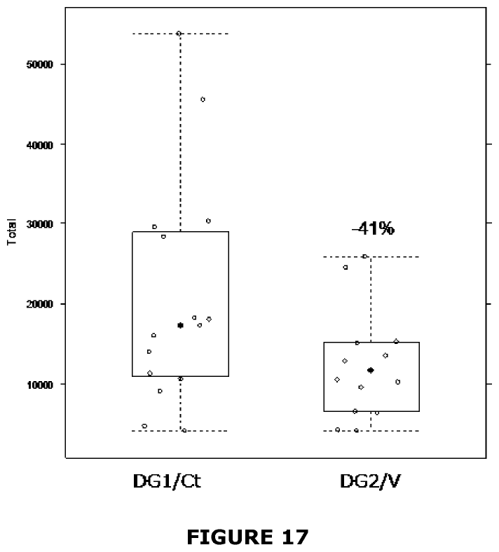

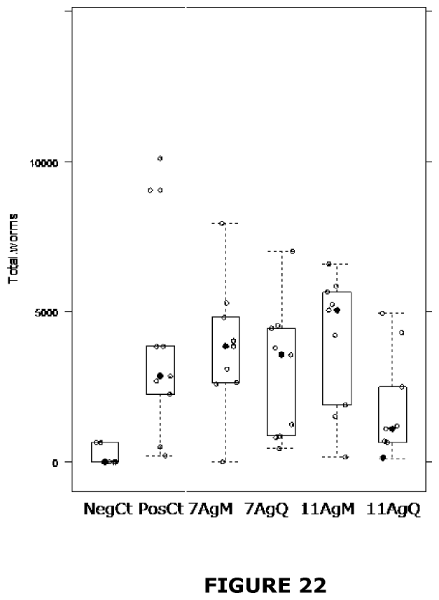

Worm burden was also reduced in animals vaccinated with the vaccine compositions of the present invention. Total adult worms as well as total male and female worms were significantly reduced as a result of vaccination in sheep, deer and calves with seven and eleven antigens as compared to the control groups. Again, the worm burden reduction was similar for each vaccine, 7AgV or 11AgV, with 65% reduction in total worm burden seen in the first sheep trial in animals vaccinated with 7AgV (see FIG. 4); a 54% reduction using the 7AgV in sheep versus 46% reduction using the 11AgV in the second sheep trial (see FIG. 6); a 69% reduction in sheep worm burden was seen in a further sheep trial using the 11AgV (see FIG. 10); a 41% reduction in worm burden was observed in deer vaccinated with the 11AgV (see FIG. 17); and a 56% reduction seen in calves vaccinated with the 11AgV (increasing to a 71% reduction when an outlier animal was removed) (see FIG. 22). The reduction in adult worm count with the vaccine compositions of the invention was significant and sufficient to prove efficacy of a vaccine composition comprising from seven to eleven antigens.

It is also envisaged that the vaccine composition of the present invention will also be effective using antigenic fragments of T. circumcincta EN, AK, ODC, SRS-2, MIF-2, aldolase, and GAPDH, as would be understood by a skilled worker.

Antigenic fragments of the optional recombinant antigens (viii)-(xi), above, may also be used in the vaccine composition of the invention.

An antigenic fragment is understood to mean a fragment of any one or more of antigens (i)-(xi) that will have effective antigenic properties, i.e. will result in the generation of antibodies that will recognise and bind to the corresponding worm proteins. A skilled worker is easily able to test fragments of antigens (i)-(xi) to determine antigenicity, i.e. antibody response, by performing enzyme-linked immunosorbent assay (ELISA) against immune or naive sheep saliva and serum using standard procedures.

Alternatively, species-specific recombinant homologs of the T. circumcincta antigens, or fragments of recombinant homologs of the T. circumcincta antigens, can be used that have at least 70%, at least 75%, at least 80%, at least 85%, at least 90%, at least 95% or at least 98% sequence identity thereto as would be understood by a skilled worker. Such recombinant homologs can be identified and produced using known technology.

In addition, corresponding native antigens can be used in place of or together with the recombinant antigens disclosed herein, as would be understood by a skilled worker, bearing in mind the problems associated with native antigens, as discussed in the background section.

In a further embodiment, the composition or vaccine composition of the invention comprises the recombinant T. circumcincta antigens (i) EN of SEQ ID NO: 1, (ii) AK of SEQ ID NO:2, and (iii) ODC of SEQ ID NO:3; (iv) SRS-2 of SEQ ID NO: 4; (v) MIF-2 of SEQ ID NO:5; (vi) Aldolase of SEQ ID NO:6; (vii) GADPH of SEQ ID NO:7, or antigenic fragments thereof, together with a veterinary acceptable carrier or diluents.

The composition or vaccine composition may further comprise one or more of the recombinant T. circumcincta antigens selected from the group consisting of:
(viii) ICL of SEQ ID NO:8;
(ix) MS of SEQ ID NO:9;
(x) CHT of SEQ ID NO: 10; and
(xi) GST of SEQ ID NO: 11;
referred to in Table 2, below, or antigenic fragments thereof.

In one embodiment the composition or vaccine composition of the invention may comprises antigens comprising at least 70% sequence identity to SEQ ID NOS: 1-11.

TABLE 2
Teladorsagia circumcincta Antigen Protein Sequences

Teladorsagia circumcincta enolase (EN) protein sequence (SEQ ID NO: 1)
MPITKIHARQIYDSRGNPTVEVDLYTDKGVFRAAVPSGASTGVHEALELRDKDKKVHHGKGVLKAVA
NINEKIAPALIAKNFCVTQQRDIDQFMLALDGTENKSNLGANAILGVSLAVAKAGAVHKGMPLYKYLA
ELAGVSKVILPVPAFNVINGGSHAGNKLAMQEFMILPVGASSFHEAMRMGSEVYHHLKAEIKKRYGL
DATAVGDEGGFAPNIQDNKEGLDLLKTAIDLAGYTGKISIGMDVAASEFYKEGKYDLDFKNPKSDPS
KWLTGDQLAALYQTFIKEYPVVSIEDAFDQDDWDNWGKLKAATNIQLVGDDLTVTNPKRIRQAIDK
KSCNCLLLKVNQIGSVTESIEAAKLSRSNGWGVMVSHRSGETEDTFIADLVVGLATGQIKTGAPCRS
ERLAKYNQLLRIEEELGKDAVYAGQNFRNPVAAAGAPVPYPDPLEPRAAAHHHHHH
Teladorsagia circumcincta arginine kinase (AK) protein sequence (SEQ ID NO: 2)
MRGSHHHHHHGMASMTGGQQMGRDLYDDDDKDHPFTMSVPPEIIKKIEDGYQTLQNAKDCHSLL
KKYLTKEVVDQLKDKKTKLGATLWDVIQSGVANLDSGVGVYAPDAEAYTLFKPLFDPLIQDYHNGFS
PSQKQPATDLGEGKTAQLVDLDPEGKYINSTRVRCGRSLQGYPFNPCLTEANYLEMEAKVKKIFENIS
DPELQGTYYPLDGMTKEVQNQLIKDHFLFKEGDRFLQAANACRYWPKGRGIFHNKNKTFLVWANEE
DHLRIISMQNGGNVGQVLERLIKGVKIIQAQAPFSRDDRLGWLTFCPSNLGTTVRASVHIRLPKISAK
PDFKKICDDLKLQIRGIHGEHSDSEGGVYDISNKARLGLTEFEAVKQMYDGVKHLIELEKKA
Teladorsagia circumcincta ornithine decarboxylase (ODC) protein sequence
(SEQ ID NO: 3)
MTMITQMELIGDSKVAIADGEVDAISMCQEIAHSYDQDNIDDAFMLVDLDVIFERFLLWKREMPMIE
PFYAVKCNTDRVLVRTLAALGAGFDCASREEIDIVMDMGVRAEKIIYANPCKTRSFITHAKEKNVSM
MTFDSVEELAKIAHLHPDAKMILRIAVSDPTARCPLNLKFGVDPVTKAPHLLVHAKELGVNVIGISFHV
GSGCNDPTAFREALTHARHLTELGRGLGFDMNLVDLGGGYPGTLQQTSFEDIAAVIRSAVDEFLPPE
FGVRLIAEPGRFFAAAPFTLVCNIIHATEVSAEKITKRPEDVDHRGFMYYVNDGVYGSFNCILFDHVDP
VGAPLFDEIVEEYPSTIWGPTCDSLDKIEDQKMMRMMSVGEWIVYQNMGAYTCSASTTFNGFQRPN
AVYVISRKNWARISTSPNV
Teladorsagia circumcincta seryl tRNA synthetase (SRS-2) protein sequence
(SEQ ID NO: 4)
MVLDMDLFREEKGGNPELIRSSQRQRYSDPSIVDKVIELDQAWRKERFLLDVLNRQKNVLSKAIGEKM
KKKEAQGTDENVDGSIISQLESLKMEDLSALTVTQIKKLRVLLDEKMNDTKVSMEQLEEDRHQSLIQIG
NIVHHSVPVSDDEANNRVERTHGDITSRKKYSHVDLVVMIDGFDGERGTTVAGGRGYFLKGPLVFLE
QAIIQLALQKLGEKGFTPLYTPFFMRKEVMQEVAQLNQFDEELYKVCGKGSEMLGDSSVDEKYLIATS
EQPIAAFHRNEWIKESDLPIKYAGISTCFRQEVGSHGRDTRGIFRVHQFEKVEQFVICSPLNNESWKM
FDEMIFNAEECCQLLGIPYQIMCIVSGELNNAASKKLDLEAWFPGSGAFRELVSCSNCTDYQARRLKV
RYGMTKKMDGEVPFVHMLNATMCATTRVLCALLENYQTEDGITVPEVLHPFMPEKYRTFIPFVKPAPI
DEEVKKKGGKAAAGAPVPYPDPLEPRAAAHHHHHH
Teladorsagia circumcincta macrophage migration inhibitory factor 2 (MIF-2)
protein sequence (SEQ ID NO: 5)
MPMVRVATNIPDKDVPPNFEERLTDILAESMNKPRTRIAVEVYAGQRIMHGGVRNPVVIIKIESIGALE
PDKNIRHTERVTQLCQDVLHVPKDKVVISYFDLAPTNVGFGGTTVAAATVAAAGAPVPYPDPLEPRAA
AHHHHHH
Teladorsagia circumcincta aldolase protein sequence (SEQ ID NO: 6)
MRGSHHHHHHGMASMTGGQQMGRDLYDDDDKDHPFTMASYSQPPPKEKEDELRGIANAIVAPGK
GILAADESTGSMDKKMKGIGTENTEEQRRKYRQLLFTASPEMSKHISGVIMFHETFYQKCDDGTRFV
DALKKQGIIPGIKVDKGVVPMAGTVGEGTTQGMDDLNARCAQYKKDGAQFAKWRCVHKISATTPS
HMALVEIAEVLARYASICQQNGLVPIVEPEILPDGEHDIDRCRKITETVLSYCYRALNDHHVYLEGTLL
KPNMVTAGQAFKGKKPSHDEIALATITALQRSVPAAVPGVVFLSGGQSEEDATLNLNAMNKLDTKKP
WALTFSYGRALQASCMSKWGGKDENVKDAQAVFMQRAQANSLAALGKYSGDPNADKAASQSLFV
ANHAY
Teladorsagia circumcincta glyceraldehyde-3-phosphate dehydrogenase
(GAPDH) protein sequence (SEQ ID NO: 7)
MGKAKVGINGFGRIGRLVLRAAVEKDTVEVVSVNDPFINIDYMVYLFKYDSTHGRFKGSVSHEGDHL
VVSKEGKSTHRIKVHNSKDPAEIPWGADGAEYIVESTGVFTTVDKASAHLKGGAKKVVISAPSADAP
MFVMGVNNETYNASNNHIISNASCTTNCLAPLAKVIHDNFGIIEGLMTTVHATTATQKTVDGPSGKL
WRDGRGAAQNIIPAATGAAKAVGKVIPALNGKLTGMAFRVPTPDVSVVDLTCRLEKPASMDDIKKVI
KSASEGAMKGILGYTEDQVVSTDFLSDTHSSIFDAGACISLNPHFVKLISWYDNEYGYSHRVVDLLT
YIASKA
Teladorsagia circumcincta isocitrate lyase protein sequence (ICL)
(SEQ ID NO: 8)
MAQAAKNWYQVVKAAPKGRFQGIKRDYQVEDVLKLRGSVEIEYTLATRGANKLWQLLHTEPFVPAL
GAQTGNQAVQMVRAGLKAIYLSGWQVAADANTAGDMYPDQSLYPANSGPELCRRINRSLRRADQI
EAVEAEDYLAQRDWYAPIVADAEAGFGGALNCFELMKSYIEAGAAGVHFEDRLGSEKKCGHMGGKV
LIPTAQHIRHLNAARLAADVCGAPTIVVARTDAESSRLLTSDVDERDHPYIDRQAGRTVEGFHRLKDS
TALQYCIDRAINYAPYCDLIWMETSHPTIADAREFAEGVRKVYPDKMFAYNCSPSFNWKQHLSPTQL
EKFQKELGALGFKYQFITLAGFHANSYSMFDLARNYKEKGMLAYSSLQQQEFAAEQHGYSAVKHQR
EVGTGYFDHISNAVTGGQSSTTALAGSTEEAQFHTATASSEDEEILAAAGAPVPYPDPLEPRAAAHH
HHHH
Teladorsagia circumcincta malate synthase (MS) protein sequence
(SEQ ID NO: 9)
MTLTAPMAAGDEKILTPDALRFIKDLNKKFDGKRRELLKKRQQVQIEINDGVYFPDFSSETAHLREDM
GWKGSEIPQDLQDRRVEITGPTDRKMVINALNSGANVFMADFEDSNTPSWRNQLEGQVNLYDAVR
DNISYMHPTTKKEYTLNQKVAVLNVRPRGWHLPEKHVLIHNKPTSGSLFDFGLFVYHNAKALKDKGS
GPYFYLPKLQNAEEAKLWAEVFAYSEDRLDLPRGTIKCTVLIEHLLATFQMNEIIYALKDHIVGLNCGR
RDYIFSYIKTFQNHRKFLLPDRFQIGMTSPFMRAYSLLCIKTCHQRGIHAMGGMAAQIPIKNDDVANS
KALALVHQDKEREATDGHDGTWVAHPGLVPIARKVFDDCMPSANQIEKQLQSFFVTNQELTAIPEGT
RTDHGFRHNISVTLGYLDSWLRGVGCVPLYNLMEDAATAEISRSQLWQWLRHDARLEDGRTIDAQL
VKQTIAAETERRLIRAGSVVSRLPEAAELLEKFALEEHMSDFLTLDAYDKLVSEGHAAAGAPVPYPDPL
EPRAAAHHHHHH
Teladorsagia circumcincta chitinase (CHT) protein sequence (SEQ ID NO: 10)
MVAETPVRRKVFINSAIAFVRQWDFDGIDIDWEYPSGPADVRNYASFISELRQACEAEATSSQKPRL
LVTAAVSAGESTIDAGYDVPAIADHLDFILLMNYDFHGAWSTETGFNSPLYAREDMRESEKVWNID
WSANHWHQKGMAKEKIIIGIPTYGRGWTLKDKSNITVGAEGSPAKITPYTQEAGVASFYEFCEMLAT
GATRYWSSEQQVPYLVQGDQWWSYDDEESIANKMAWIKRNKYGGAFVWTLDFDDFNAKCSNSD
GQLYPLISIIAKELGGVTIPKVSTCSTWGNMDDVVDKGLSIAAAGAPVPYPDPLEPRAAAHHHHHH
Teladorsagia circumcincta glutathione S-transferase (GST) protein sequence
(SEQ ID NO: 11)
MVHYRLLYFDGRGRAEVARQLFALANQEYVDVRITHEEWPKHKPEMPFGQLPVLDVDGKLLGQSHAI
NRYLARQFGFAGKSPFEEALVDAFADQYRDFYTEAQPYLYAVWGFVKGDVNALENEKFAPARDKFFN
LMTKHLKASKSGFLVGDSVTWADLQLAELATFTEKYATLYVGFPEVKAHSEKVRSIPEIKKRIETRKN
TPFAAAGAPVPYPDPLEPRAAAHHHHHH

The composition or vaccine composition of the invention optionally includes an adjuvant.

The term “adjuvant” as used herein refers to an agent used to enhance the immune response of the immunised host to the immunising composition.

Suitable adjuvants for the vaccination of farmed or wild ruminant animals include but are not limited to oil emulsions such as Freund's complete adjuvant, Freund's incomplete adjuvant, squalane or squalene; mineral gels such as aluminium hydroxide, aluminium phosphate, calcium phosphate, calcium phosphate and alum; surfactants such as hexadecylamine, lysolecithin and methoxyhexadecylglcerol; polyanions such as dextron sulphate and carbopol; peptides such as muramyl dipeptide and dimethylglycine; or other adjuvants including QuilA, lipopolysaccharide, montanide, lipovant, bacterial flagellin, adjuvant 65, imiquimod, gamma inulin, guardiquimod, etc.

A preferred adjuvant is Montanide or Quil A.

The composition or vaccine composition of the invention optionally includes a carrier.

The composition or vaccine composition of the invention may include a carrier selected from, but not limited to, solgel (a chitin based slow release compound), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes, cochleate delivery vehicles, etc.

The composition or vaccine composition of the present invention can be given to an animal before any infection is detected to act as a preventative or can be given as a treatment to infected animals.

The composition or vaccine composition of the present invention is preferably in a form for administering to an animal via subcutaneous or intramuscular injection.

The composition or vaccine composition of the present invention will be formulated for subcutaneous or intramuscular administration as a parenterally acceptable aqueous solution which is pyrogen-free and has a suitable pH, isotonicity and stability.

The composition or vaccine composition may contain salts, buffers, adjuvants or other substances which are desirable for improving the efficacy of the composition as would be understood by a skilled worker.

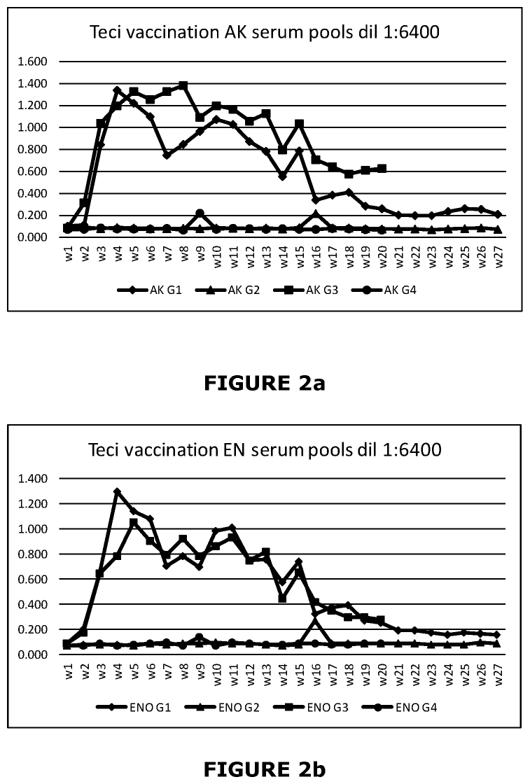

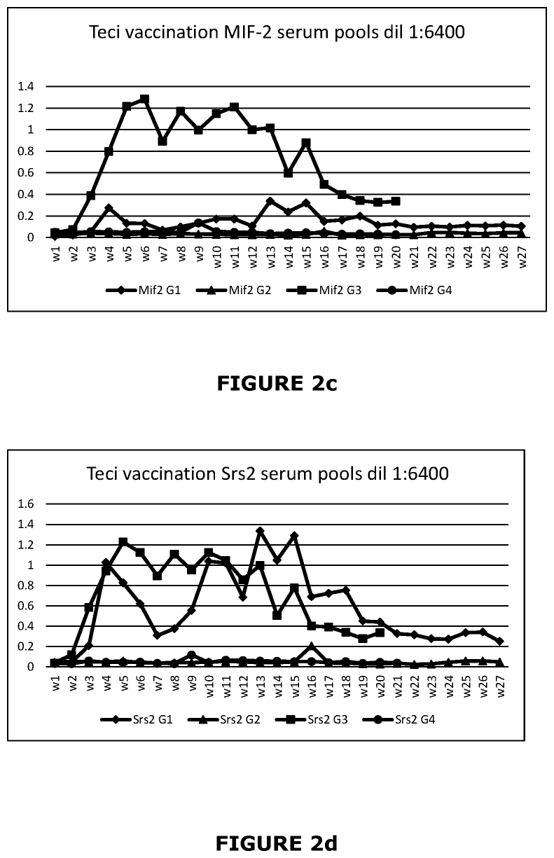

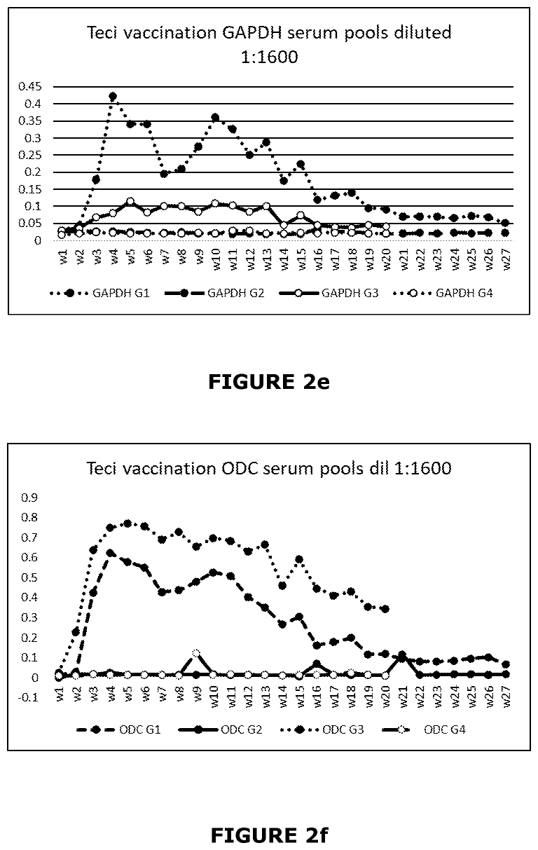

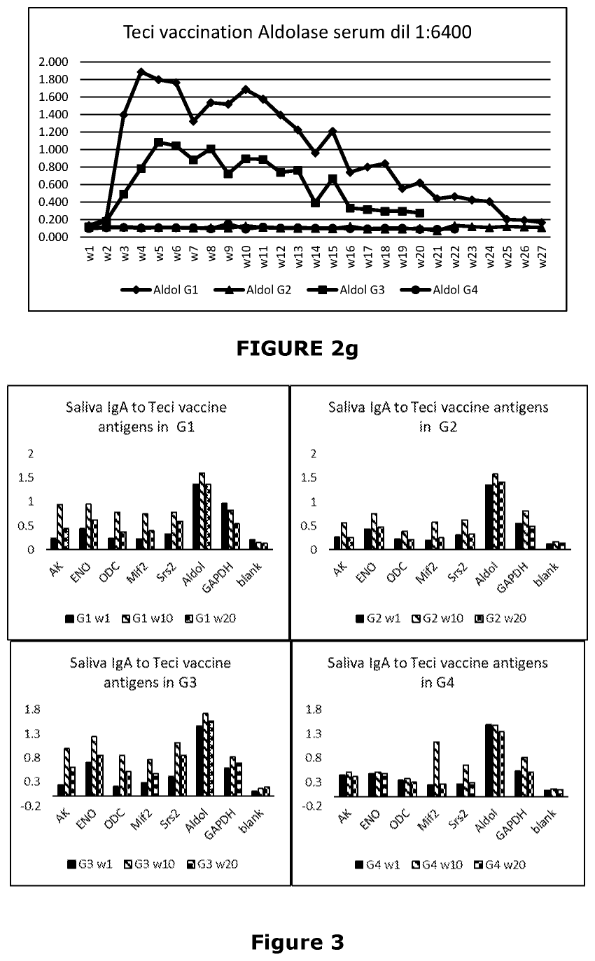

The composition or vaccine composition of the invention will be administered to an animal in a therapeutically effective amount, i.e. an amount that results in an immunologic response such as the production of desirable antibodies. As can be seen in the examples, sustained antibody responses to vaccines comprising seven and eleven antigens has been demonstrated in sheep, deer and calves (see FIGS. 2a-2g, 8, 11, 13-15, 18 and 19). These antibody responses correlated with reduced worm burden in sheep, deer and calves evidencing the efficacy of the vaccines. Based on the results, it is expected that the vaccine would be efficacious in other ruminants.

Typically, the amount of antigens administered to an animal is between about 30 μg-250 μg of each antigen, preferably between about 50 μg-200 μg, more preferably between about 75 μg-150 μg of each antigen.

The composition or vaccine composition of the invention can be administered as a single or multiple dose of a therapeutically effective amount. Preferably, the composition or vaccine composition is administered twice, with a primary immunisation given followed by a booster 2-8 week later, preferably 3 weeks later. In some ruminants, a second booster may be required around 4-8 weeks after the first booster depending on antibody levels as would be understood by a skilled worker.

Additional doses can be administered as required to treat or prevent infection as would be understood by a skilled worker.

The composition or vaccine composition of the invention can be administered with anthelmintic agents such as levamisole, morantel, oxfendozole, monepantel and/or ivermectin to increase the overall FEC reduction rates and worm burden of the treated animals at the time of vaccination.

The composition or vaccine composition of the invention can also be administered with other vaccine treatments commonly administered to ruminants such as clostridial diseases (including pulpy kidney, tetanus, malignant oedema, black disease and black leg); bovine viral diarrhoea (BVD); footrot; leptospirosis; salmonella; scabby mouth, etc.

The composition or vaccine composition of the present invention is formulated to treat or prevent nematode worm infection in farmed or wild ruminants, particularly in sheep, cattle, goats, deer, buffalo, bison, camelids and llamas. Preferably the composition or vaccine composition of the invention is formulated to treat or prevent nematode worm infection in farmed animals including cattle, sheep, goats and deer, especially in young animals, less than one year old. In one aspect, the animal may be less than 6 months old. In another aspect, the animal is at least 3 months old.

The term “comprising” as used in this specification and claims means “consisting at least in part of”. When interpreting statements in this specification, and claims which include the term “comprising”, it is to be understood that other features that are additional to the features prefaced by this term in each statement or claim may also be present. Related terms such as “comprise” and “comprised” are to be interpreted in similar manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This invention may also be used to broadly consist in the parts, elements and features referred to or indicated in the specification of the application, individually or collectively, and any or all combinations of any two or more said parts, elements or features, and where specific integers are mentioned herein which have been equivalents in the art to which this invention relates, such known elements are deemed to be incorporated herein as if individually set forth.

The invention will now be described by way of the following representative methods and examples which are provided to further illustrate the subject matter to which the invention relates. The use of any and all examples, or exemplary language (e.g., “such as” or “including”) provided herein, is intended solely for the purposes of better describing the invention. The presence of examples and the use of exemplary language does not limit the scope of the invention as disclosed herein unless specifically otherwise indicated. No language used in the entirety of the disclosure of this application should be interpreted as indicating that any particular element or feature pertaining to the invention and as disclosed herein is essential to the practice of the invention, unless explicitly stated. For example, the skilled worker will be able to modify the quantities of reagents and processing times in the methods and processes as exemplified, as known in the art, according to the inventive concepts disclosed herein. Such modifications are considered to be within the scope of the present invention.

### EXAMPLES

### Trial 1

Immunisation of sheep with a vaccine composition comprising T. circumcincta enolase (EN), arginine kinase (AK), ornithine decarboxylase (ODC), seryl tRNA synthetase (SRS-2), macrophage migration inhibitory factor 2 (MIF-2), aldolase and glyceraldehyde-3-phosphate dehydrogenase (GAPDH).

### Materials and Methods

### Preparation of Antigen

Recombinant AK, EN, ODC, SRS-2, MIF-2, aldolase and GAPDH were purified as described (Han et al., 2012; Umair et al 2013 a, b). Proteins were individually identified on gels stained with Coomassie Blue to confirm size and solubility.

### Recognition of Vaccine Antigens by Immune Lambs

Prior to the start of the trial, mucosal and systemic antibody responses against recombinant EN, MIF-2, aldolase, GST, CHT, ICL, MS, and GAPDH were evaluated by performing enzyme-linked immunosorbent assay (ELISA) against immune or naive sheep saliva and serum using standard procedures (Umair et al., 2021a; 2021b; 2020a; 2020b; 2017; 2016; 2023 in press).

### Animals

Use of experimental animals was approved by the AgResearch Animal Ethics Committee. 36 male Romney cross lambs (~3 months old) were purchased from Ballantrae Farm, AgResearch Ltd and were transported to Grasslands Large Animal facility, drenched, weighed and tagged. Animals were randomly divided into four groups with nine animals in each group. The average group weight was the same in all groups before the start of the trial. Animals were housed and fed indoors. Animals were divided into following groups:
Group 1: Vaccine in Quil A and sol gel (VacQuil)
Group 2: Quil A control group (Quil A in sol gel) (CtQuil)
Group 3: Vaccine in Montanide ISA 71VG (1:2 ratio) (VacMon)
Group 4: Montanide ISA 71VG control group (CtMon)

### Vaccination Trial

Each animal in the vaccinated groups received 50 μg of each antigen by SC injection. Animals of groups 3 and 4 received vaccine or adjuvant three times at 3 weeks intervals whereas the animals of groups 1 and 2 received vaccine or adjuvant at week 0 and 6. This trial was initiated as an indoor trial but as some of the animals did not adjust to the indoor management system, soon after the third vaccination animals were moved to AgResearch's Aorangi farm. They were grazed in parasite-free paddocks and 4 weeks after the third vaccination, challenged with 12,000 L3 T. circumcincta over 3 days. At 5 weeks post-infection, the animals of group 3 and 4 were killed, but as there were no differences in the egg output of the animals of groups 1 and 2, these groups were re-challenged with 20,000 L3 T. circumcincta over 3 weeks and killed 3 weeks after the second parasite challenge. Abomasa were collected from the killed animals for parasitology study (adult male and female worm counts).

### Immune Assays and Parasitology

Antibody levels in serum and saliva were measured by ELISA. Eggs per gram faeces (EPG) were counted using the modified McMaster method in which each egg counted represented 50 eggs per gram faeces (Lyndal-Murphy, 1993). Adult worms were recovered from the abomasa in a volume of 7 litres and 10% was used to measure worm counts, male to female ratio and worm lengths. Worm lengths were performed using ImageJ software.

### Functional Activities of Recombinant Enzymes

Purified recombinant AK, EN, ODC, SRS-2, MIF-2, aldolase and GAPDH were incubated in immune serum from animals of Quil A vaccine group at 25° C. for an hour and enzyme assays were performed to determine if antibodies in serum can inhibit the function of these enzymes. Recombinant enzymes were also incubated in naive serum to serve as controls. AK, EN, ODC, SRS-2, MIF-2, aldolase and GAPDH assays were performed according to the protocol described (Umair et al., 2013a; Han et al., 2013; Umair et al., 2013b).

### Results and Discussion

Antibodies in saliva and serum from naturally immune sheep strongly reacted with the 7 recombinant proteins demonstrating that the immune host can recognise these antigens.

### Antibody Responses

The serum IgG antibody response against recombinant AK, EN, ODC, SRS-2, MIF-2, aldolase and GAPDH was measured by ELISA. Enzyme-specific IgG levels were significantly higher in Quil A and Montanide ISA 71VG vaccine groups compared to their respective control groups (FIGS. 2a-g) in pooled serum at a dilution of 1:6400 or 1:1600. The salivary antibodies (IgA) also increased in the Quil A and Montanide ISA 71VG vaccine groups following the vaccination compared with their respective controls (FIG. 3).

### Parasitology and Adult Worm Count

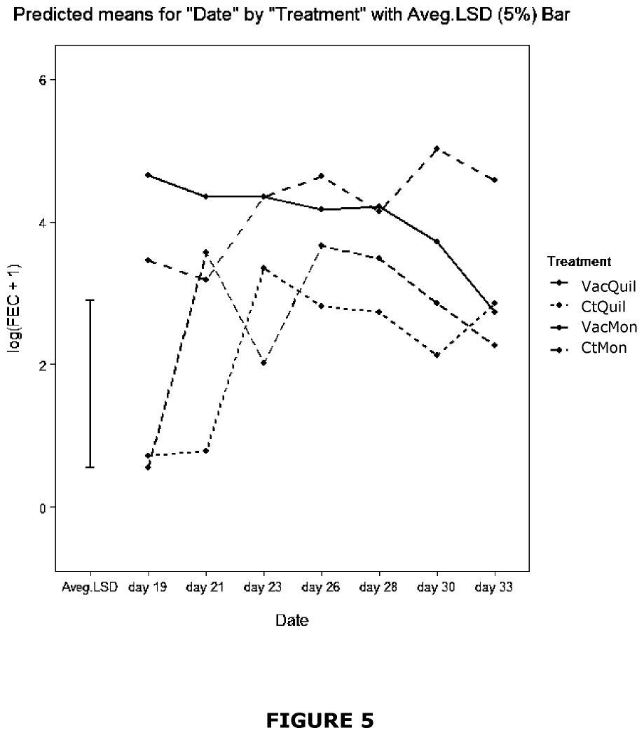

The results of this trial were outstanding with a significant reduction in adult worm numbers (both male and female) in the animals of group 3 compared to the control animals of group 4 (P<0.05) (FIG. 4), with the difference in the faecal egg count approaching significance (P=0.06) (FIG. 5). The VacMon group (group 3) had higher weight gains compared to the control group (group 4) (~1.5 kg, although this was not statistically significant-results not shown).

### Discussion

This trial demonstrated that immunization of 3-4 month-old sheep with a cocktail of seven recombinant proteins induced levels of protection against T. circumcincta. The recombinant 7 antigen vaccine in Montanide ISA 71VG adjuvant (VacMon) resulted in significant reduction in the adult worm reduction (both male and female). VacMon also resulted in reduced faecal output compared to control (P=0.06). When the same cocktail of recombinant proteins was used with another adjuvant, Quil A, there were no differences in faecal egg output and adult worm count between VacQuil and CtQuil. However, two animals from the CtQuil group had to be removed from the trial because of lack of weight gain, which could have affected the lack of significant reduction in the VacQuil group.

### Conclusion

The results of this trial were very promising and show for the first time that recombinant antigens can be made that elicit significant immune responses when injected into sheep.

Further experiments with larger group size would be useful to conclusively establish if these antigens are protective and suitable vaccine targets.

### Trial 2

Immunisation of sheep with a vaccine composition comprising 7, 8 or 11 T. circumcincta recombinant antigens.

### Materials and Methods

### Animals and Experimental Design

Use of experimental animals had been approved by the AgResearch Animal Ethics Committee. 90 newly-weaned female Romney cross lambs (approximately 3 months old) were purchased from private farms and transported to AgResearch Ltd Aorangi Farm, drenched, weighed and tagged. Animals were randomly divided into five groups (InfCt, 7AgV, 8aAgV, 8bAgV and 11AgV) with 15 animals in each group. In addition, 15 animals were used in a non-infected control group (-Ct). The average group weight was identical in all groups before the start of the trial. Animals were fed on pasture with a history of no sheep grazing for at least last three years. These paddocks were grazed by cattle prior to and during the experiment.

The experiment was designed to test if recombinant Teladorsagia antigens given in different combinations can improve the vaccine efficacy compared to the previous trial. Animals were divided into following groups:
Group 1: non-infected control group (-Ct)
Group 2: infection control group (PostCt)
Group 3: 7 antigens vaccine in Montanide ISA 71VG (7AgV)
Group 4: 8 antigens in Montanide ISA 71VG (8aAgV)
Group 5:8 antigens in Montanide ISA 71VG (8bAgV)
Group 6:11 antigens in Montanide ISA 71VG (11AgV)

### Vaccine Antigens and Formulations

### Seven Antigen Vaccine (7AgV)

(i) Arginine kinase (AK)
(ii) Enolase (EN)
(iii) Ornithine decarboxylase (ODC);
(iv) seryl tRNA synthetase (SRS-2);
(v) macrophage migration inhibitory factor 2 (MIF-2);
(vi) aldolase;
(vii) glyceraldehyde-3-phosphate dehydrogenase (GAPDH)
   Eight Antigen Vaccine (8aAgV)
(ix) malate synthase;
(viii) ICL;
(iii) ODC;
(iv) SRS-2;
(v) MIF-2;
(vi) aldolase;
(vii) GADPH;
and (x) Cht.
   Eight Antigen Vaccine (8bAgV)
(i) AK;
(ii) EN;
(iii) ODC;
(iv) SRS-2;
(viii) ICL;
(x) Cht;
(ix) malate synthase;
and (xi) GST.

### Eleven Antigen Vaccine (11AgV)

(i) AK
(ii) EN
(iii) ODC
(iv) SRS-2
(v) MIF-2
(vi) aldolase
(vii) GADPH; and
(viii) isocitrate lyase (ICL);
(ix) malate synthase;
(x) chitinase (Cht); and
(xi) glutathione S-transferase (GST)

Recombinant antigens were purified as described before (Han, et al. 2012; Umair, et al. 2013 a, b). Proteins were individually identified on gels stained with Coomassie Blue and size and solubility confirmed.

Recombinant proteins were expressed in E. coli (see Appendix 2). Each antigen was formulated in equal amounts of adjuvant (Montanide ISA71 VG). Each animal received a 3 ml dose for each vaccination and received a total of 3 vaccinations at 3 week intervals. 50 μg of each antigen was administered on each occasion.

### Vaccination Trial

Animals in groups 7AgV, 8aAgV, 8bAgV and 11AgV received a subcutaneous dose (3 ml per animal) of 50 μg of each antigen formulated in Montanide ISA71 VG. Three vaccinations were performed weekly for three weeks (day 0, day 7 and day 21). The control groups were not injected with adjuvant or vaccine.

Two weeks following the last vaccination, all animals except -Ct group were infected with an oral dose of 2000 L3 T. circumcincta larvae 3× a week for 4 weeks. -Ct animals served as non-infected controls to establish potential pasture contamination.

All animals were killed 6 weeks post-infection, and abomasa were collected for parasitology. Total numbers of adult male and female T. circumcincta were counted, and the number of eggs within the adult female worms were also counted.

### Results

The results of this trial were at the highest end of our expectations, wherein treatment with 7AgV and 11AgV significantly reduced the adult worm number (both male and female) when compared with the PosCt group (FIG. 6) (P<0.05). In addition, the difference in the faecal egg count between the 7AgV and PosCt groups was also significant (P<0.05) (FIG. 7a) and each antigen resulted in a significant antibody response (FIG. 7b). The 7AgV group had higher weight gains compared to the InfCt group (~1.5 kg, although this was not statistically significant—results not shown).

### Discussion

This trial demonstrated the efficacy of the 7 and 11 recombinant vaccine against the mucosal browser nematode parasite T. circumcincta in young animals. Vaccination with a combination of recombinant parasite antigens induced a significant increase in antibody levels and a significant reduction in egg counts and worm burdens. Importantly, vaccine efficacy was achieved in young lambs that are highly susceptible to parasite infections under field conditions. Here, immunization of young sheep (approximately 3 months old) with a cocktail of recombinant proteins induced levels of protection against T. circumcincta.

### Conclusion

The present vaccine comprising the seven core antigens (7AgV) with or without additional multiple recombinant antigens resulted in a prototype vaccine that showed efficacy in young sheep under stringent field conditions where animals were exposed to a number of environmental stresses. Interestingly the two eight antigen vaccines (8aAgV and 8bAgV) that did not include the 7 core antigens were not effective, evidencing that the 7AgV core antigens are essential for efficacy of the vaccine of the present invention.

### Trial 3

Immunisation of sheep with a vaccine composition comprising 11 T. circumcincta recombinant antigens.

### Materials and Methods

### Animals and Experimental Design

Use of experimental animals had been approved by the AgResearch Animal Ethics Committee. 80 newly weaned lambs, ~3 months old females, were grazed outdoors at Grasslands for one week and were introduced to pellets, lucerne pellets and lucerne chaff. The lambs were then moved indoors and for one week provided with cut grass, pellets, lucerne pellets and lucerne chaff to help them to adapt to the indoor environment. To complete the adaption to the indoor environment, the animals were only fed pellets, lucerne pellets and lucerne chaff. 60 animals that had adapted to the indoor environment were selected for the trial.

The experiment was designed to determine the efficacy of Teladorsagia vaccine against single- or triple-resistant field strains of Teladorsagia; to determine the efficacy of Teladorsagia vaccine when administered intra-muscularly; and to determine the efficacy of Teladorsagia vaccine when given in low dose (20 μg/antigen/animal/vaccine, instead of the usual dose of 50 μg/antigen/animal/vaccine).

Animals were divided into the following groups:

| | Number | |
|---|---|---|
| Group | (n) | Treatment |
| LG1/Ct | 10 | Control group, challenged only with susceptible strain |
| LG2/V-S | 10 | Vaccinated and challenged with susceptible lab strain. |
| LG3/VIM-S | 10 | Vaccinated intramuscularly and challenged with susceptible |
| | | lab strain |
| LG4/VLC-S | 10 | Vaccinated with a low concentration (20 μg) of each antigen |
| | | and challenged with susceptible lab strain |
| LG5/V-1R | 10 | Vaccinated and challenged with strain resistant to one |
| | | anthelmintic |
| LG6/V-3R | 10 | Vaccinated and challenged with strain resistant to three |
| | | anthelmintics |

### Vaccine Antigens and Formulations

### Eleven Antigen Vaccine (11AgV)

In this group, the vaccine comprised eleven antigens, namely:
(i) AK
(ii) EN
(iii) ODC
(iv) SRS-2
(v) MIF-2
(vi) aldolase
(vii) GADPH; and
(viii) isocitrate lyase (ICL);
(ix) malate synthase;
(x) chitinase (Cht); and
(xi) glutathione S-transferase (GST)

Recombinant antigens were purified as described before (Han et al. 2012; Umair et al. 2013 a, b). Proteins were individually identified on gels stained with Coomassie Blue and size and solubility confirmed.

Recombinant proteins were expressed in E. coli (see Appendix 2). Each antigen eas formulated in equal amounts of adjuvant (Montanide ISA 71VG). Each animal received a 3 ml dose for each vaccination and received a total of 3 vaccinations at 3 week intervals. 50 μg of each antigen was administered on each occasion.

### Vaccination Trial

Animals in groups 2, 3, 5 and 6 received a subcutaneous dose (3 ml per animal) of 50 μg of each antigen formulated in Montanide ISA 71VG. Animals in group 4 received a low dose of only 20 μg of each antigen. Three vaccinations were performed weekly for three weeks (day 0, day 7 and day 21). Animals in the control group 1 were not injected with adjuvant or vaccine.

Two weeks after the third vaccination all lambs were orally dosed daily with 3000 L3 T. circumcincta larvae (as shown in the Table above) for five days. All the animals were slaughtered 8-10 weeks post-challenge and the abomasa collected for parasitology.

### Results

### Antibody Responses

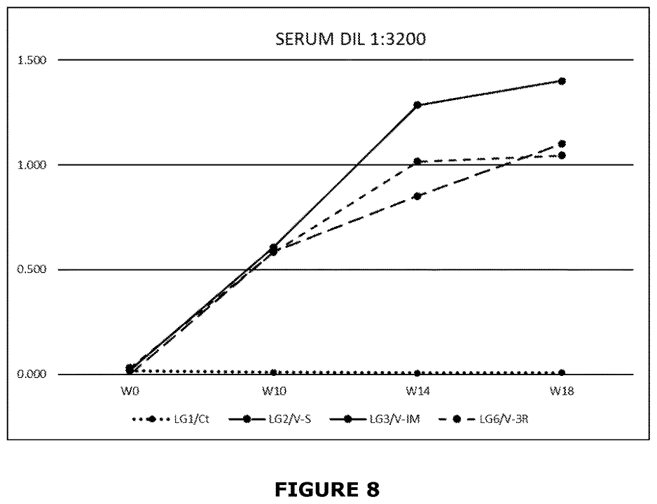

Vaccination induced significantly higher antibody response in vaccine groups compared to that of the control (FIG. 8). Serum samples of LG2/V-S, LG3/V-IM and LG6/V-3R were tested at weeks 0, 10, 14 and 18, and compared to the controls (LG1/Ct). All three vaccinations induced significantly higher antibodies compared to the controls, however there was no differences in the antibody titres between the vaccinated groups. The antibody titres increased after each vaccine and peaked following the last vaccination.

### Faecal Egg Counts

Vaccination resulted in significantly lowering the faecal egg count in LG6/V-3R and resulted a 70% reduction compared to control (FIG. 9). Although vaccination did result in reduction of 26% and 29% in FEC for LG2/V-S, LG3/VIM-S, it was not statistically significant. No other vaccine group had significantly lower faecal egg counts compared to the control group. Saliva samples for this trial were not analysed but the saliva samples from animal trial 1 showed significant increase in IgA antibody in the vaccine groups compared to the control group.

### Adult Worm Count

When the adult parasites (both male and female) were counted from the abomasa collected from the slaughtered animals, LG3/VIM and LG6/V-3R had significantly fewer adult worms in them compared with LG1/Ct (FIG. 10). Vaccination did not have any detrimental effects on animals' body weights and there were no significant differences in the lambs' body weights between vaccinated and control groups.

### Discussion

Vaccination significantly reduced faecal egg count in the treatment group infected with triple resistant Teladorsagia strain. Vaccination resulted in a significant reduction of adult worm counts in LG3/VIM-S and LG6/V-3R. Vaccination delivered intramuscular provided better protection as compared to the SC route. Interestingly, the lower antigen concentration was not protective.

### Conclusion

The present vaccine comprising eleven recombinant antigens (11AgV) resulted in a prototype vaccine that showed efficacy in young sheep against multiple resistant worms under stringent field conditions where animals were exposed to a number of environmental stresses.

### Trial 4

Immunisation of calves with a vaccine composition comprising 11 T. circumcincta recombinant antigens.

### Materials and Methods

### Animals and Experimental Design

Use of experimental animals had been approved by the AgResearch Animal Ethics Committee. 30 newly-weaned male Friesian calves, ~3 months old, were grazed on parasite-free pastures at AgResearch Aorangi farm. The calves remained outdoors thoughout the course of the trial. The calves were separated into two groups of 15 animals each.

The experiment was designed to establish the proof of concept that a recombinant Teladorsagia vaccine can protect against Ostertagia infection in cattle.

The animals were divided into the following groups:

| | Number | |
|---|---|---|
| Group | (n) | Treatment |
| CG1/Ct | 15 | Control group, challenged only |
| CG2/V | 15 | Vaccinated with 11 antigens and |
| | | challenged. |

### Vaccine Antigens and Formulations

### Eleven Antigen Vaccine (11AgV)

In this group, the vaccine comprised eleven antigens, namely:
(i) AK
(ii) EN
(iii) ODC
(iv) SRS-2
(v) MIF-2
(vi) aldolase
(vii) GADPH; and
(viii) isocitrate lyase (ICL);
(ix) malate synthase;
(x) chitinase (Cht); and
(xi) glutathione S-transferase (GST)

Recombinant antigens were purified as described before (Han et al. 2012; Umair et al. 2013 a, b). Proteins were individually identified on gels stained with Coomassie Blue and size and solubility confirmed.

Recombinant proteins were expressed in E. coli (see Appendix 2). Antigens were formulated in an equal amount of adjuvant (Montanide ISA 71VG). Each animal received a 4 ml dose for each vaccination and received a total of 3 vaccinations at 3 week intervals. 150 μg of each antigen was administered on each occasion.

### Vaccination Trial

The control group was not vaccinated (CG1/Ct) and the calves of the vaccination group (CG2/V) received a subcutaneous dose (4 ml per animal) of 150 μg of each antigen formulated in Montanide ISA 71VG three times at three-weekly intervals. Two weeks after the third vaccination all animals were challenged with a total of 15,000 L3 Ostertagia ostertagi larvae administered over 4 days. Adult worms from abomasa were recovered and counted following slaughter.

### Results

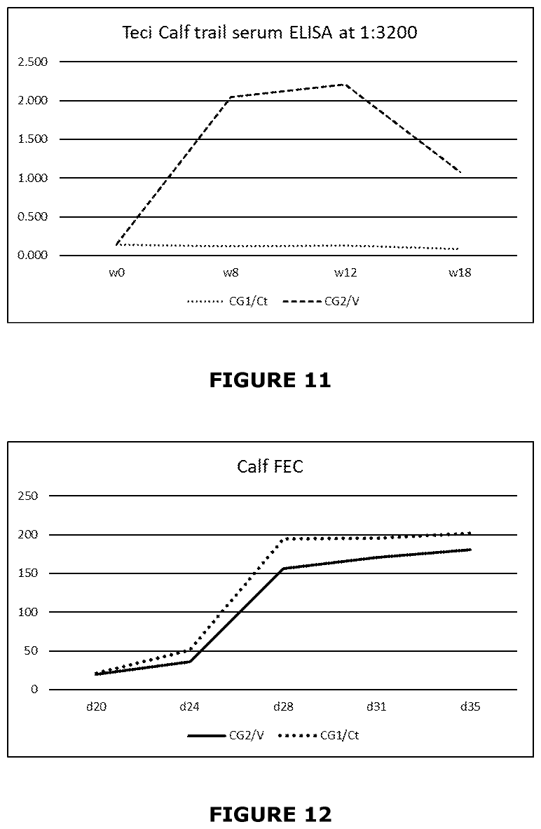

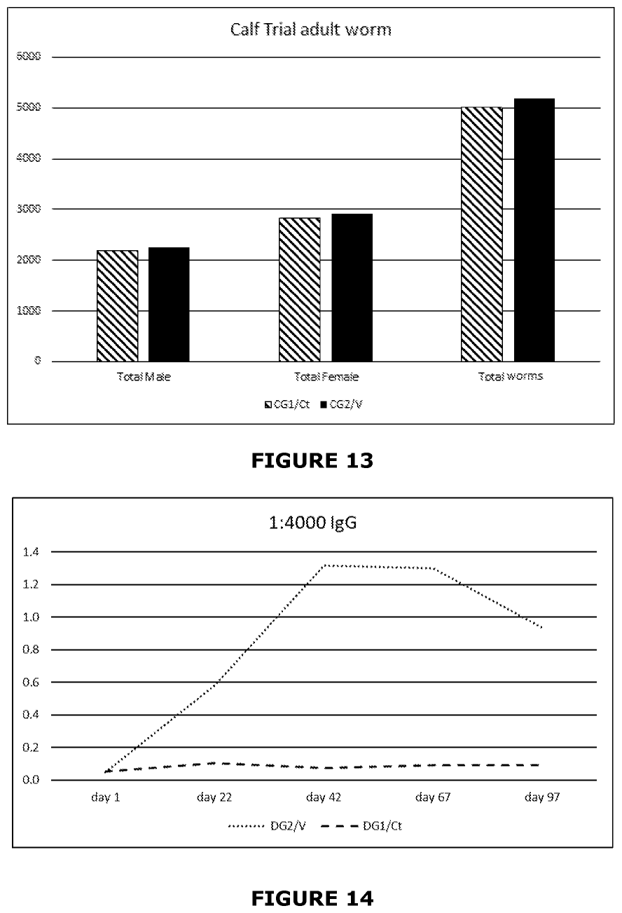

Although the vaccination did result in the production of significantly higher antibody levels (FIG. 11) there was no significant reduction in faecal egg counts (FIG. 12) or adult worm burden (FIG. 13) between the two groups, nor was there any difference in the weekly weight-gain between the two groups. This appears to show that antibodies produced against antigens of sheep T. circumcincta fail to protect against O. ostertagi infection and that T. circumcincta vaccine is possibly species-specific and may not function against other parasites of the same family or genus.

### Discussion

It was a little surprising to note that the vaccine did not even partially work against the cattle O. ostertagi infection although the two parasites appear very similar. There are no O. ostertagi published gene sequences in the databases, which makes it very hard to compare the homology between the T. circumcincta and O. ostertagi gene sequences. The likely reason for it not working is because the epitopes of the recombinant proteins are different in two parasite species even though most of the gene sequences are likely to be closely related. Another likely reason the vaccine is ineffective is the adjuvant choice. Our previous experience with parasite vaccine shows that vaccine efficacy is highly dependent on the choice of adjuvant. The Montanide ISA 71VG adjuvant worked for the sheep might not work for cattle. Another cattle trial was planned with a different adjuvant/s (see TRIAL 6, below).

### Trial 5

Immunisation of deer with a vaccine composition comprising 11 T. circumcincta recombinant antigens.

### Materials and Methods

### Animals and Experimental Design

Use of experimental animals had been approved by the AgResearch Animal Ethics Committee. 30 male red deer, ~3-4 months old, were grazed on standard pastures throughout the course of the trial at AgResearch Farm at Invermay, the deer were separated into two groups of 15 animals each.

The experiment was designed to establish the proof of concept that a recombinant Teladorsagia vaccine can protect against Ostertagia type infection in deer.

The animals were divided into the following groups:

| | Number | |
|---|---|---|
| Group | (n) | Treatment |
| DG1/Ct | 15 | Control group, natural challenge only |
| DG2/V | 15 | Vaccinated with 11 antigens and naturally |
| | | challenged. |

### Vaccine Antigens and Formulations

### Eleven Antigen Vaccine (11AgV)

In this group, the vaccine comprised eleven antigens, namely:
(i) AK;
(ii) EN;
(iii) ODC;
(iv) SRS-2;
(v) MIF-2;
(vi) aldolase;
(vii) GADPH;
(viii) isocitrate lyase (ICL);
(ix) malate synthase;
(x) chitinase (Cht); and
(xi) glutathione S-transferase (GST).

Recombinant antigens were purified as described before (Han et al. 2012; Umair et al. 2013 a, b). Proteins were individually identified on gels stained with Coomassie Blue and size and solubility confirmed.

Recombinant proteins were expressed in E. coli (see Appendix 2).

The control group was not vaccinated (DG1/Ct) and the animals of the vaccination group (DG2/V) received a subcutaneous dose (3 ml per animal) of 150 μg of each antigen formulated in Montanide ISA 71VG three times at three-weekly intervals.

### Vaccination Trial

The control group was not vaccinated (DG1/Ct) and the treated group was vaccinated three times at three-weekly intervals with 100 μg of each antigen formulated in Montanide ISA 71VG.

Compared to the artificial parasite challenge used in the lamb and calf trials described above, these animals were naturally challenged by grazing them on standard deer pasture where contamination with deer parasites is expected to be relatively high. All animals were weighed, bled, saliva and faecal sampled once a month. The trial was terminated 8-10 weeks following the last vaccination and samples collected for analysis.

### Results

### Antibody Responses

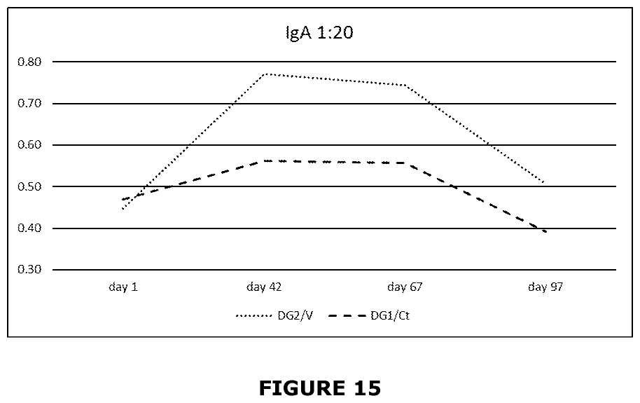

Vaccinated resulted in significantly higher antibody titre (IgG) in the serum samples (FIG. 15) and saliva (FIG. 16) samples of all the animals of the vaccinated group compared to those of the control group. The antibody titres peaked after the third vaccination but started to drop three weeks after the last vaccination.

Faecal egg and adult worm counts Vaccination resulted in reduction in faecal egg output in the vaccine group compared to the control group (FIG. 16) and it appears the vaccine resulted in ~49% reduction. The vaccination resulted in 41% reduction in the adult worm reduction in DG2/V (1 outlier was taken out) (FIG. 17).

### Discussion

The findings to this first deer trial were very encouraging and it appears that a recombinant Teladorsagia vaccine partially protects against Ostertagia type infection in weaned red deer. Parasites are the biggest problem in farmed deer and the current anthelmintics are not effective. Ostertagia type worms are the most important parasites in the NZ farmed deer. More experiments are required to increase vaccine efficacy and optimise the vaccine concentration and route of delivery.

### Conclusion

The present vaccine comprising eleven recombinant antigens (11AgV) resulted in a prototype vaccine that showed efficacy in young deer under stringent field conditions where animals were exposed to a number of environmental stresses.

### Trial 6

Immunisation of calves with a vaccine composition comprising 7 or 11 T. circumcincta recombinant antigens in two different adjuvants (Montanide ISA 61VG and Quil A/Sol gel).

### Materials and Methods

### Animals and Experimental Design

Use of experimental animals had been approved by the AgResearch Animal Ethics Committee. 69 male calves, about 5-6 months of age, were purchased and taken to AgResearch Aorangi farm two weeks prior to the outdoor trial. All animals except negative controls were pink-tagged and randomly divided into one of the 7 groups (see below).

This experiment was designed to determine the efficacy of Teladorsagia vaccine in cattle against Ostertagia ostertagi infection using a number of antigen combinations; and to determine the efficacy of Teladorsagia vaccine in cattle against O. ostertagi infection with antigens combination formulated in two different adjuvants, including a new Montanide adjuvant (Montanide ISA 61VG) as Montanide ISA 71VG previously didn't work in cattle (see Trial 4, above).

Calves were divided into the following groups:

| | Number | |
|---|---|---|
| Group | (n) | Treatment |
| −Ct | 6 | No treatment, no infection control |
| InfCt | 9 | Infection Control group |
| Vac1 (7AgM) | 9 | 7 antigens in Montanide ISA 61VG |
| Vac2 (7AgQ) | 9 | 7 antigens in Solgel 1 (chitosan + |
| | | QuilA) |
| Vac3 (11AgM) | 9 | 11 antigens in Montanide ISA 61VG |
| Vac4 (11AgQ) | 9 | 11 antigens in Solgel 1 (chitosan + |
| | | QuilA) |

### Vaccine Antigens and Formulations

### Seven Antigen Vaccine (7AgV)

(i) Arginine kinase (AK)
(ii) Enolase (EN)
(iii) Ornithine decarboxylase (ODC)
(iv) seryl tRNA synthetase (SRS-2);
(v) macrophage migration inhibitory factor 2 (MIF-2);
(vi) aldolase; and
(vii) glyceraldehyde-3-phosphate dehydrogenase (GAPDH).

### Eleven Antigen Vaccine (11AgV)

(i) AK;
(ii) EN;
(iii) ODC;
(iv) SRS-2;
(v) MIF-2;
(vi) aldolase;
(vii) GADPH;
(viii) isocitrate lyase (ICL);
(ix) malate synthase;
(x) chitinase (Cht); and
(xi) glutathione S-transferase (GST).

Recombinant antigens were purified as described before (Han et al. 2012; Umair et al. 2013 a, b). Proteins were individually identified on gels stained with Coomassie Blue and size and solubility confirmed.

Recombinant proteins were expressed in E. coli (see Appendix 2). Antigens were formulated in equal amounts of adjuvant (either Montanide or QuilA as set out in the Table above). Each animal received a 4 ml dose for each vaccination and received a total of 3 vaccinations at 3 week intervals. 150 μg of each antigen was administered on each occasion.

### Vaccination Trial

The candidate vaccine (150 μg of each purified antigen) was administered as a Subcutaneous Injection (SC) in the neck. 2-3 ml was given using an 18 gauge needle on each of 3 occasions at 3 weekly interval for Montanide vaccine groups (Vac1 and Vac3) and twice at 6 weekly interval for Solgel vaccine groups (Vac2 and Vac4). Vaccination sites were monitored and checked twice post vaccination in the same week and weekly afterwards. All animals were bled and weighed weekly and saliva sampled fortnightly. Faecal samples were collected monthly prior to the parasite infection and 2 times a week post infection starting from day 18 to count egg per gram of faeces.

Animals were grazed on ‘parasite free’ new pastures, weekly shifts with break fence front & back to avoid reinfection once the animals start to shed the eggs in the faeces following challenge. All animals except -Ct were challenged with 15,000 O. ostertagi larvae. All animals were slaughtered on day35 post infection and abomasa collected for adult worm and L4 recovery.

### Results

### Antibody Response

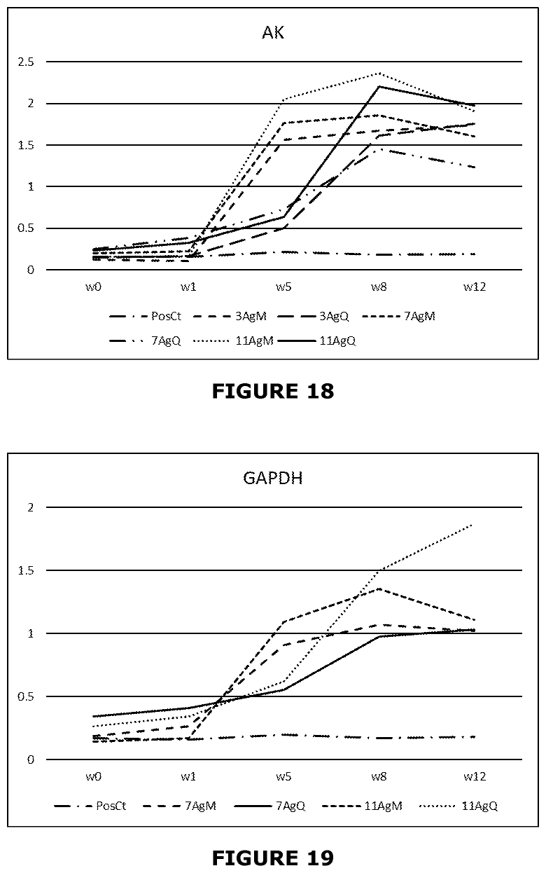

Vaccination resulted in the production of antigen specific antibodies in all the vaccine groups. The Montanide adjuvant groups received three shots of each vaccine over 3 weekly interval whereas the QuilA/Sol gel adjuvant groups received two vaccination. The antibody titre of the QuilA/Sol gel animals was equal or higher than that of Montanide although the former animals received only two vaccine shots. The antibody response was generally high in all the vaccinated animals (Two examples shown in FIGS. 18 and 19). FIG. 19 shows the antibody response in 4 vaccine groups when vaccinated with recombinant GAPDH along with other antigens (as described in the methodology, above). Animals vaccinated with the 11AgV had highest antibody response at week 12 (averaged value presented in FIG. 19) compared with the control animals. Antibody levels were measured by ELISA on sera diluted 1:4000 at optical density 450 nm.

### Faecal Egg Counts

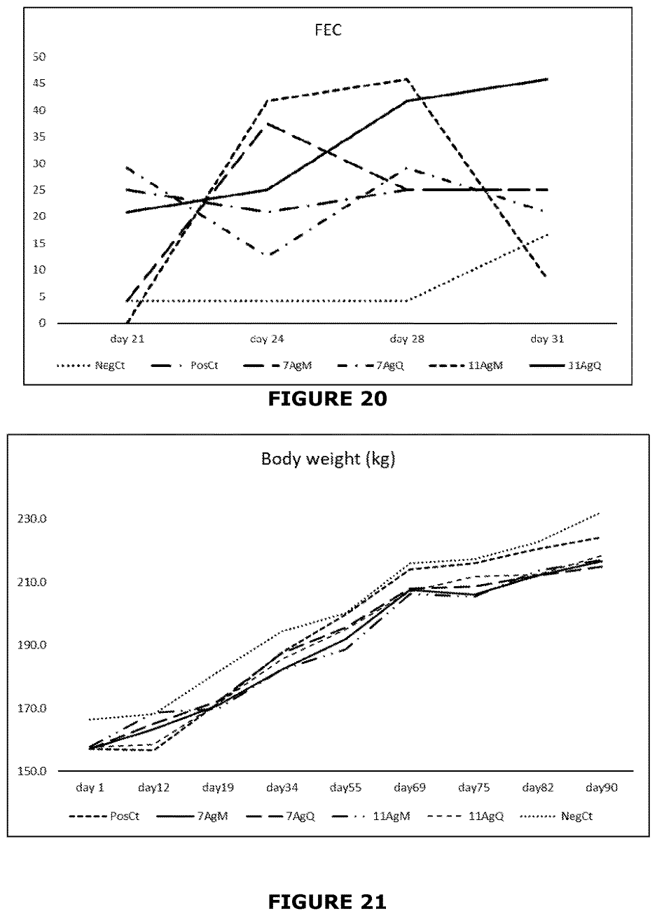

Faecal samples from all the animals were collected twice a weekly for two weeks. No vaccine resulted in significant reduction in the FEC compared to the control group. There was no difference in the faecal egg output between different vaccine groups. Similarly, there was no difference in the faecal egg output between Montanide or Quil A/Solgel groups (FIG. 20).

### Body Weight

The average body weight in all the vaccine and control groups is shown in FIG. 21. There were no significant differences between the weight gains in various groups and the average weight gain between all the groups was similar although the positive control group animals had an average of 3-4 kg more weight gain than any of the vaccine group but it was not statistically significant.

### Adult Worm Count

The adult worm count was the most interest aspect of this trial and vaccination resulted in significant reduction of adult worm counts in 11AgV (QuilA/Sol gel) group. The adult worm reduction in this group was by 56% compared to the control group. There was an outlier animal in this group and the reduction was increase to 71% if that animal was taken out of the analysis. There were no significant differences in any other vaccine groups compared to the control group (FIG. 22).

### Arrested Larvae Recovery and Counts

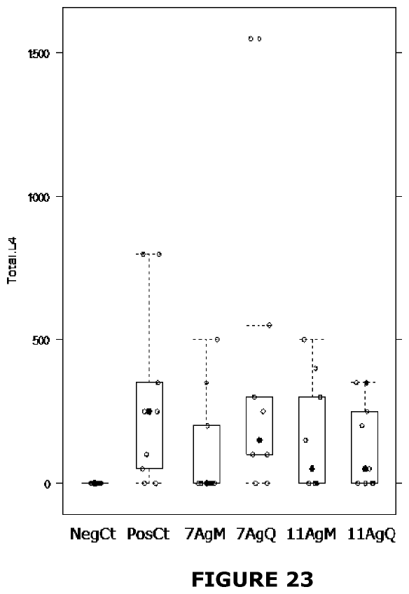

Arrested L4 were recovered from abomasa of all the vaccine group animals by incubating each abomasum at 37° C. in in 1 L water containing 30 ml hydrochloric acid and 10 g pepsin. Vaccination resulted in significant reduction of L4 in the abomasa of animals vaccinated with the 7AgV (in Montanide adjuvant) and with the 11AgV (in QuilA/Sol gel adjuvant) where the percentage reduction was 59% and 54% respectively. When one outlier each was removed from both of the groups, the percentage reduction of L4 was around 70% in both of the groups when compared with the control group (FIG. 23).

### Discussion

This trial evidenced for the first time that a vaccine comprising recombinant Teladorsagia antigens showed efficacy against O. ostertagi infection in calves. Although the vaccine failed to reduce faecal egg output in any of the vaccine groups, there was significant reduction in the adult worm number (both male and female O. ostertagi) by around 70%. O. ostertagi, fourth stage larvae go into arrested development in the mucosa of the abomasa and months later (when the conditions outside the host are favourable) develop into the adult worms and complete the life cycle. Vaccination with the vaccines if the present invention significantly reduced the number of L4 being arrested by around 70%, which means there will be fewer larvae to develop into the adult worm in the spring season. This aspect of the anti-O. ostertagi vaccine has very important implications for the livestock industry. In New Zealand, most of the larvae on pasture die during the winter because of the extreme cold temperatures and frost and the arrested larvae within the host act as a reservoir and in spring develop into adult worms and start infecting the pastures. This vaccine could potentially get rid of most of the arrested L4 population means that there will be no or very few eggs shed on the pasture in the subsequent season.

The vaccine resulted the generation of high antibody titres in both QuilA/Solgel and Montanide ISA 61VG vaccine groups compared to the control group. QuilA/Solgel groups (7AgV and 11AgV) performed better than three vaccine groups in Montanide adjuvant in the sense that they had similar antibody levels but these animals received only two vaccine shots compared to the three shots of the Montanide groups. Furthermore, animals in the Montanide groups had severe and adverse vaccine site reaction compared to that of QuilA groups. Animals vaccinated with QuilA/Solgel had significantly fewer and smaller lumps compared to that of the Montanide. Data indicate that the more the number of antigens (regardless of the adjuvant) the bigger the size of the lump. Histology of the samples taken from the vaccine site shows calcification and eosinophilic infiltration.

### Conclusion

The present vaccine comprising seven or eleven recombinant antigens (7AgV, 11AgV) resulted in a prototype vaccine that showed efficacy in young calves under stringent field conditions where animals were exposed to a number of environmental stresses. Both adjuvants (Montanide ISA 61VG and QilA/Solgel) were useful. We also showed that the new Montanide adjuvant (ISA 61VG) could successfully be used in calves whereas the original Montanide adjuvant (ISA 71VG) was successfully used in sheep and deer. This is important information for vaccine formulation for target animals.

### Trial 7

Immunisation of calves with a vaccine composition comprising 7 or 11 T. circumcincta recombinant antigens.

### Materials and Methods

### Animals and Experimental Design

Approval for the use of experimental animals was sought from the AgResearch Animal Ethics Committee. 35 Jersey calves, ~4 months old, were brought to Aorangi farm two weeks before the trial. Animals were drenched to remove existing worm burden, weighed and saliva sampled. Animals were fed on parasite-free pasture throughout the trial. The animals were randomly allocated into four treatment groups.

This experiment was designed to determine the efficacy of Teladorsagia vaccine in cattle against Ostertagia ostertagi infection using a number of antigen combinations; and to determine the efficacy of Teladorsagia vaccine in cattle against O. ostertagi infection with antigens combination formulated in Quil A and chitosan based slow release formulation.

Calves were divided into the following groups:

| | Number | |
|---|---|---|
| Group | (n) | Treatment |
| NegCt | 2 | No treatment, no infection control |
| PosCt | 11 | Infection Control group |
| Vac1 (7AgQ) | 11 | 7 antigens in Quil A and Solgel |
| Vac2 (11AgQ) | 11 | 7 antigens in Solgel 1 (chitosan + |
| | | QuilA) |

7 weeks post second vaccination, the animals of Group 1 (NegCt), and 8 animals each from the remaining groups were slaughtered for adult worm recovery and tissue collection. The 3 remaining animals from each group were grazed for another 6 months. Serum samples were collected every fortnight to access the antibody levels, and the duration of protection was determined.

### Results

### Faecal Egg Counts

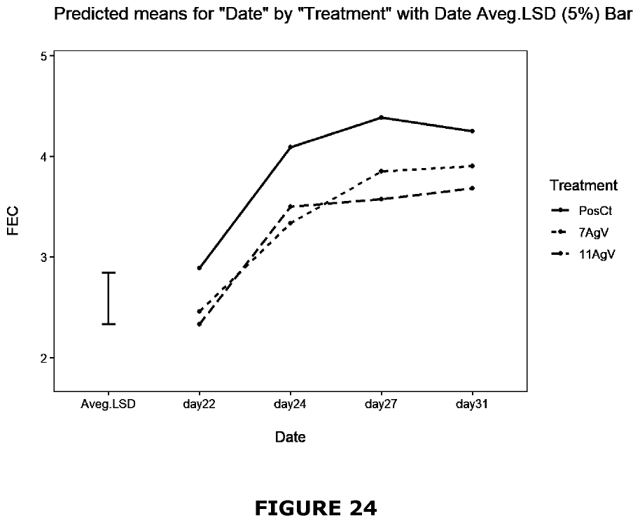

The faecal egg output of animals of the vaccinated groups from day 21 to day 31 post-infection was significantly lower than either of the positive control groups (FIG. 24). The 11AgV group had significantly lower egg output at every sampling time point, whereas the 7AgV group was significantly reduced at two sampling time points. Animals of the negative control group (not shown in FIG. 24) did not get the parasite challenge and, hence, had zero faecal egg output.

### Adult Worm Counts

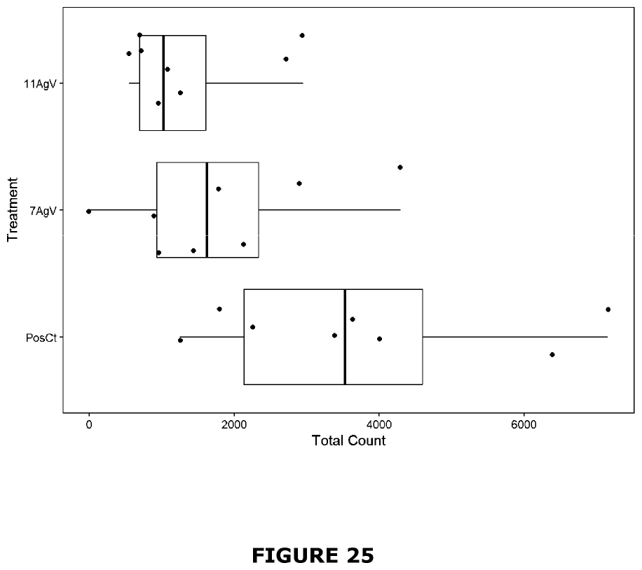

Total adult worms and total male and female worms were significantly reduced in the 11AgV animals compared to the control group (FIG. 25). The 11 AgV group showed an overall reduction of 67% in the adult worms compared with PosCt.

### Arrested Larval Recovery and Counts

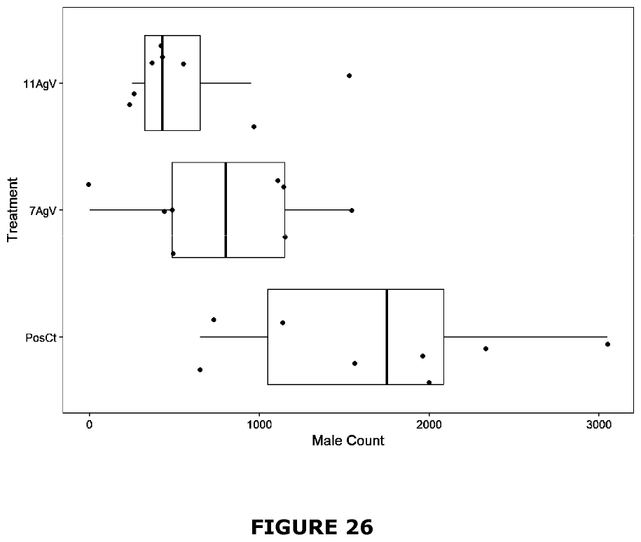

Arrested L4 were recovered from abomasa of calves of all the vaccine group animals by incubating each abomasum at 37° C. in 1 L water containing 30 ml hydrochloric acid and 10 g pepsin. Vaccination significantly reduced L4 in the abomasa of both the 7AgV and 11AgV groups (FIG. 26).

### Antibody Response

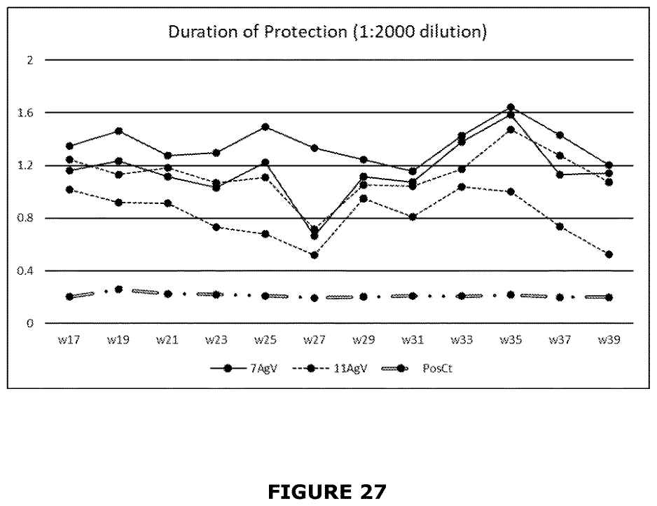

Serum samples collected weekly through the trial were tested against all 11 antigens, and serum antibody responses to vaccination were measured in individual samples. The serum samples were diluted at 1:1000, 1:2000, and 1:8000, and significantly higher antibodies were measured in all the treatment animals at all three dilutions. Saliva samples from individual animals were collected before the trial and assayed for antibodies to the CarLA to assess the level of pre-existing exposure to parasite infections. The animals were divided into control and treatment groups based on weight and the CarLA level. The CarLA levels in most of the animals were very low. Vaccination induced significantly higher serum antibody response in both treatment groups than PosCt. Serum samples from two calves from each group were collected every fortnight for seven months post-second vaccination to assess the level of antibodies. The antibody response in the 1:2000 dilution is shown in FIG. 27.

### Discussion

In this calf trial, vaccination resulted in a significant reduction in adult worm counts in both the 7Ag and the 11Ag groups. The vaccination resulted in a 67% reduction in the adult worm numbers in the 11AgV group compared with the control group. Vaccination positively impacted vaccinated calves, which excreted fewer parasite eggs in their faeces. Also, vaccination resulted in significantly higher serum antibodies in all the vaccinated animals, and the antibody titres were maintained for more than eight months after the second vaccination, suggesting that protection against O. ostertagi infection could extend months after the vaccination. The calves grazed on a good quality pasture, and the overall daily weight gain per calf was between 1.2-1.5 kg per day (no statistical differences between the vaccine and control group animals because of a small group size). These results are encouraging and will help exemplify the patent claim that the Teladorsagia vaccine protects against O. ostertagi infection in young calves in field conditions.

### Conclusion

The present vaccine comprising seven or eleven recombinant antigens (7AgV, 11AgV) resulted in a prototype vaccine that showed efficacy in young calves under stringent field conditions where animals were exposed to a number of environmental stresses. This is important information for vaccine formulation for target animals.

### General Conclusion

Teladorsagia circumcincta, also called brown stomach worm, is one of the most significant nematode parasites affecting livestock worldwide. The present recombinant Teladorsagia vaccine has shown really promising results in lambs, as shown above. In particular, the vaccine has consistently shown significant reductions in faecal egg output and adult worm counts in the young, vaccinated lambs. The present preliminary trials also show promising results against deer and cattle Ostertagia parasites. To date, there is no Teladorsagia vaccine on the market. This vaccine has a huge potential, especially against resistant T. circumcincta, where the vaccine has shown a reduction in resistant worm numbers in young vaccinated lambs, but also more broadly against corresponding Ostertagia-type worms in deer and calves.

### Appendix 1

Nematode Parasite (Teladorsagia circumcincta) Vaccine Antigen Discovery

### Overview of Antigen Selection

The rationale for this study was the discovery of proteins (genes) essential for the viability of the parasitic nematode Teladorsagia circumcincta that could be utilised as antigens for vaccine development. The discovery was approached from two directions.
1. 3 homologues of antigens were selected and correspond with those used in our earlier Haemonchus vaccine—EN, AK, ODC (see NZ 780917).
2. The remaining antigens were selected based on the literature available.

### Appendix 2

Summary of Recombinant T. circumcincta Recombinant Protein Expression and Purification.

| Recombinant | | | | Activity Assay | |
|---|---|---|---|---|---|
| antigen | Vector | Tag(s) | Purification | (NT = Not tested) | Active |
| Enolase (EN) | AY2.4 | Arabinose inducible, | Purification by | the oxidation of NADH to NAD⁺ in the second reaction | Y |
| | | C-terminal E-tag and | Ni-NTA agarose | monitored (Umair et al., 2017a) | |
| | | 6xHis | | | |
| Arginine | pET100 | | Ni-NTA agarose. | By measuring rate of utilisation of NADH, measured | Y |
| kinase (AK) | | | | spectrophotometrically at 340 nm (Umair et al. 2013b). | |
| Ornithine | pET200 | Arabinose inducible, | Ni-NTA agarose. | By measuring the ¹⁴CO₂ formation from L-[1-¹⁴C]- | Y |
| decarboxylase | | C-terminal E-tag and | | ornithine (Umair et al. 2013a) | |
| (ODC) | | 6xHis | | | |
| seryl tRNA | AY2.4 | C-terminal E-tag and | Ni-NTA agarose. | Aminoacylation of tRNA with [U-¹⁴C]serine, precipitation | NT |
| synthetase | | 6xHis | | with trichloroacetic acid (TCA) and precipitate | |
| (srs-2) | | | | captured on a glass fibre filter (adapted | |
| | | | | from methods of Taupin et al., 1997 and | |
| | | | | Weygand-Durasevic et al., 1993). | |
| aldolase | pET100 | N-terminal E-tag and | Ni-NTA agarose. | The NADH production measured colorimetrically at 450 nm | Y |
| | | 6xHis | | (Umair et al., 2021b) | |
| glyceraldehyde- | pCL476 | N-terminal 6xHistag | Ni-NTA agarose. | conversion of NAD⁺ to NADH, monitored | Y |
| 3-phosphate | | | | spectrophotometrically at 340 nm (Umair et al., 2017b) | |
| dehydrogenase | | | | | |
| (GAPDH) | | | | | |
| Isocitrate | AY2.4 | N-terminal 6xHistag | Ni-NTA agarose. | The NADH production measured colorimetrically | Y |
| lyase (ICL) | | | | at 450 nm (Umair et al., 2020a) | |
| Malate | AY2.4 | N-terminal 6xHistag | Ni-NTA agarose. | | N |
| synthase (MS) | | | | | |
| macrophage | AY2.4 | C-terminal E-tag and | Soluble, Ni-NTA | Tautomerization of the coloured dopachrome | NT |
| migration | | 6xHis | agarose. | methyl ester (Swope et al., 1998). | |
| inhibitory | | | | | |
| factor (mif-2) | | | | | |
| Chitinase (CHT) | pET200 | N-terminal 6xHis | Ni-NTA agarose. | | N |
| Glutathione | AY2.4 | N-terminal 6xHis | Ni-NTA agarose | Increase in absorbance measured at 340 nm upon | Y |
| S-transferase | | | | conjugation of thiol group of glutathione | |
| (GST) | | | | and CDNB (2020b) | |

### REFERENCES

Lyndal-Murphy, M., 1993. Anthelmintic resistance in sheep. In: Corner, L. A., Bagust, T. J. (Eds.), Australian Standard Diagnostic Techniques for Animal Diseases. CSIRO, Melbourne, Australia, pp. 1-17.
Umair, S., Knight, J. S., Bland, R. J., Simpson, H. V., 2013a. Molecular and biochemical characterisation of arginine kinases in Haemonchus contortus and Teladorsagia circumcincta. Experimental Parasitology 134, 362-367.
Umair, S., Knight, J. S., Simpson, H. V., 2013b. Molecular and biochemical characterisation of ornithine decarboxylases in the sheep abomasal nematode parasites Teladorsagia circumcincta and Haemonchus contortus. Comparative Biochemistry and Physiology B 165, 119-124.
Umair, S., Ria, C., Knight, J. S., Bland, R. J., Simpson, H. V., 2013c. Sarcosine metabolism in Haemonchus contortus and Teladorsagia circumcincta. Experimental Parasitology 134, 1-6.
Han, K., L. Xu, et al. (2012). “Vaccination of goats with glyceraldehyde-3-phosphate dehydrogenase DNA vaccine induced partial protection against Haemonchus contortus.” Veterinary Immunology and Immunopathology 149 (3-4): 177-185.
Longhi-Browne, C. W. (2014) using Caenorhabditis elegans as a novel expression system for the generation of recombinant Teladorsagia circumcincta vaccine candidates. Abstract PhD thesis.
Swope M, Sun, H-W, Blake, P. R and Lolis E (1998) Direct link between cytokine activity and a catalytic site for macrophage migration inhibitory factor. EMBO J. 17:3534-3541
Taupin, C. M-J., Hartlein, M. and Leberman, R. (1997). Seryl-tRNA synthetase from the extreme halophile Haloarcula marismortui: Isolation, characterisation and sequencing of the gene and its expression in Escherichia coli. Eur. J. Biochem. 243:141-150
Weygand-Durasevic I., Ban, N., Jahn, D. and Soll, D. (1993). Yeast seryl-tRNA synthetase expressed in Escherichia coli recognises bacterial serine-specific tRNAs in vivo. Eur. J. Biochem. 214:869-877
Umair, S., Bouchet, C. L. G., Baten, A. 2021. Characterisation and recognition by immune hosts of a sheep nematode parasite Teladorsagia circumcincta chitinase. Journal of Veterinary Science and Medicine 9, 1-5.
Umair, S., Bouchet, C. L. G., Palevich, N., Simpson, H. V. 2021b. Teladorsagia circumcincta 1.6-Biphosphate aldolase: Molecular and biochemical characterisation, structure analysis and recognition by immune hosts. Parasitologia 1, 1-9.
Umair, S., Bouchet, C. L. G., Palevich, N., Simpson, H. V. 2020a. Characterisation and structural analysis of glyoxylate cycle enzyme of Teladorsagia circumcincta. Molecular and Biochemical Parasitology 240, 111335.
Umair, S., Bouchet, C. L. G., Deng, Q., Palevich, N., Simpson, H. V., 2020b. Characterisation of a Teladorsagia circumcincta glutathione transferase. Molecular and Biochemical Parasitology, 239, 111316.
Umair, S., Bouchet, C. B. G., Knight, J. S., Pernthaner, A., Simpson, H. V., 2017. Molecular and biochemical characterisation and immune recognition of Teladorsagia circumcincta glyceraldehyde 3-phosphate dehydrogenase (GAPDH). Experimental Parasitology 181, 40-46.
Umair, S., Bouchet, C. B. G., Knight, J. S., Pernthaner, A., Simpson, H. V., 2017a. Molecular and biochemical characterisation of Teladorsagia circumcincta enolase and its recognition by the immune host. Experimental Parasitology 172, 30-38.
Umair, S., Bouchet, C. B. G., Knight, J. S., Pernthaner, A., Simpson, H. V., 2017b. Molecular and biochemical characterisation and immune recognition of Teladorsagia circumcincta glyceraldehyde 3-phosphate dehydrogenase (GAPDH). Experimental Parasitology 181, 40-46.
Umair, S., Knight, J. S., Simpson, H. V., 2013a. Molecular and biochemical characterisation of ornithine decarboxylases in the sheep abomasal nematode parasites Teladorsagia circumcincta and Haemonchus contortus. Comparative Biochemistry and Physiology B 165, 119-124.
Umair, S., Knight, J. S., Bouchet, C. L. G., Palevich, N., Cleland, S., Grant, W., Simpson, H. V., 2022. Characterisation of macrophage inhibitory factor-2 (MIF-2) in Haemonchus contortus and Teladorsagia circumcincta. Parasitologia, 2, 338-349.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

1. A composition or vaccine composition comprising the recombinant T. circumcincta antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) aldolase; and
(vii) glyceraldehyde-3-phosphate dehydrogenase (GAPDH),
or antigenic fragments thereof, together with a veterinary acceptable carrier or diluent.

2. The composition or vaccine composition of claim 1, further comprising one or more recombinant T. circumcincta antigens selected from the group consisting of:
(viii) isocitrate lyase (ICL);
(ix) malate synthase (MS);
(x) chitinase (CHT); and
(xi) glutathione S-transferase (GST)
or antigenic fragments thereof.

3. The composition or vaccine composition of claim 2, comprising at least one, at least two, at least three or at least four of the antigens (viii) isocitrate lyase (ICL), (ix) malate synthase, (x) chitinase (Cht) and (xi) glutathione S-transferase (GST).

4. A composition or vaccine composition comprising the T. circumcincta recombinant antigens:
(i) enolase (EN);
(ii) arginine kinase (AK);
(iii) ornithine decarboxylase (ODC);
(iv) seryl tRNA synthetase (SRS-2);
(v) macrophage migration inhibitory factor 2 (MIF-2);
(vi) aldolase;
(vii) glyceraldehyde-3-phosphate dehydrogenase (GAPDH);
(viii) isocitrate lyase (ICL);
(ix) malate synthase (MS);
(x) chitinase (Cht); and
(xi) glutathione S-transferase (GST)
or antigenic fragments thereof, together with a veterinary acceptable carrier or diluent.

5. The composition or vaccine composition of any one of claims 1-4, further comprising an adjuvant.

6. The composition according to claim 5, wherein the adjuvant is selected from one or more of the group consisting of alum, Quil A, Freund's complete adjuvant, Freund's incomplete adjuvant, lipopolysacharride, monophosphoryl lipid A, montanide, lipovant, bacterial flagellin, adjuvant 65, gamma inulin, algammulin, imiquimod, guardiquimod and murimyl dipeptide.

7. The composition or vaccine composition of any one of claims 1-6, further comprising a carrier.

8. The composition of claim 7, wherein the carrier is selected from one or more of the group consisting of a chitin-based slow release compound (sol-gel), hollow mesoporous silicon nanoparticles (HMSNs), poly(d,l-lactide-co-glycolide) (PGC) nanoparticles, poly(d,l-lactic-coglycolic acid) (PGCA) nanoparticles, liposomes, virosomes and cochleate delivery vehicles.

9. A method of reducing parasitic nematode worm burden in a farmed or wild ruminant animal, said method comprising administering an effective amount of the composition or vaccine composition of any one of claims 1-8 to said ruminant animal on one or more occasions, whereby parasitic worm burden reduction is measured by a reduced faecal egg count (FEC), and/or an increase in expulsion of larvae and/or adult nematode worms.

10. A method of inducing an immune response in a farmed or wild ruminant animal to treat or protect said animal against infection by parasitic nematodes, said method comprising administering an effective amount of the composition or vaccine composition of any one of claims 1-8 to said animal on one or more occasions, wherein induction of an immune response is measured by the presence of protective antibodies against one or more specific antigens present in said composition or vaccine composition.

11. A method of stimulating or boosting acquired immunity in a farmed or wild ruminant animal to treat or protect said animal against infection by parasitic nematodes, said method comprising administering an effective amount of a composition or vaccine composition of any one of claims 1-8 to said animal on one or more occasions, wherein stimulation or a boost of said acquired immunity is measured by one or more of: the presence of protective antibodies against one or more specific antigens present in said composition or vaccine composition; an increased level of cytokines; a reduced FEC; and/or expulsion of larvae and/or adult nematodes.

12. A method of treating or preventing a nematode infection in a farmed or wild ruminant animal comprising administering an effective amount of a composition or vaccine composition of any one of claims 1-8 to said animal.

13. A use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase; and (vii) glyceraldehyde-3-phosphate dehydrogenase (GAPDH) or antigenic fragments thereof, in the manufacture of a composition or vaccine composition for reducing nematode parasitic worm burden in a farmed or wild ruminant animal.

14. A use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase; and (vii) glyceraldehyde-3-phosphate dehydrogenase (GAPDH) or antigenic fragments thereof in the manufacture of a composition or vaccine composition for stimulating or boosting acquired immunity in a farmed or wild ruminant animal to treat or protect said animal against infection by parasitic nematodes.

15. A use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase; and (vii) glyceraldehyde-3-phosphate dehydrogenase (GAPDH) or antigenic fragments thereof, in the manufacture of a composition or vaccine composition for treating or preventing a nematode infection in a farmed or wild ruminant animal.

16. A use of the recombinant T. circumcincta antigens (i) enolase (EN), (ii) arginine kinase (AK), (iii) ornithine decarboxylase (ODC), (iv) seryl tRNA synthetase (SRS-2), (v) macrophage migration inhibitory factor 2 (MIF-2), (vi) aldolase, and (vii) glyceraldehyde-3-phosphate dehydrogenase (GAPDH), or antigenic fragments thereof, in the manufacture of a composition or vaccine composition for inducing an immune response in a farmed or wild ruminant animal to treat or protect said animal against infection by parasitic nematodes.

17. A use as claimed in any one of claims 13-16, wherein the composition or vaccine composition further comprises one or more of antigens (viii) isocitrate lyase (ICL), (ix) malate synthase, (x) chitinase (Cht) and (xi) glutathione S-transferase (GST), as defined in claim 2.

18. A method as claimed in any one of claims 9-12, or a used as claimed in any one of claims 13-16, wherein the farmed or wild ruminant animal is selected from the group consisting of sheep, cattle, goat, deer, buffalo, bison, camelids and llamas.

19. A method or use as claimed in claim 18, wherein the farmed or wild ruminant animal is a young animal, less than one year old.

20. A method or use as claimed in claim 19, wherein the farmed or wild ruminant animal is less than 6 months old.

21. A method as claimed in any one of claims 9-12 and 18-20, or a use as claimed in any one of claims 13-17, wherein the parasitic nematode worms are selected from one or more of the group consisting of Trichostrongylus colubriformis, Haemonchus contortus, Haemonchus placei, Ostertagia (Teladorsagia) circumcincta, Cooperia curticei, Nematodirus spathiger, Trichostrongylus axi, Trichostrongylus vitrinus, Ostertagia ostertagia, Cooperia oncophera, Nematodirus brasiliensis, Dictyocaulus eckerti, Strongylus vulgaris, Toxascaris vitolorum, Nematodirus filicollis, Ashworthius sidemi, Mecistocirrus digitatus, Bunostomum trigonocephalum, Trichuris discolour and Toxacara vitulorum.
